SEQUENCE LISTING (59 sequences)

SEQ ID NO: 1 - DNA, 11097 bp, Foot-and-mouth disease virus
  source 1..11097: mol_type = genomic DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcatatgtaatacgactcactatagggttgaaagggggcgctagggtctcacccctagcatgccaacgacagctcctacgtcgcactccacactaacgtttgtgtgcgcgcgggaaccgatggacttttgttcacccacctacagttggactcacggcaccgcgtggccattttagctgggttgtgcggacgaacactgcttgcgcatctcgcgtgaccggttagtactcttaccactatccgcctacttggtcgttagcgctgtcctgggcactcttgttgggggctgttcaacgctctacggtctcccctgcgtaacagactacggtgttggggccgcttcgtgcgagccgatcgcttggtgtgcctcggctgtcgcccgaagcccgcctttcaccccccccccccccccccccctaggttttaccgtcgttcccgacgttaatggggaaacaaccacaagcttaacaccgtcttgcccgacgtaaaagggctgcaaccaaaaagcttgtgccgcctttcccggcgttaatgggaggtaaccacaagacaaaccttcacccggaagtaaaacggcaacttcacacagttttgcccgttttcgtgagaaatgggccgtcaacgcacgaaacgcgccgtcgcttgaggaggacttgtacaaacacgatctatgcaggtttccacaactgacacaaaccgtgcaacttgaaaccccgcctggtctttccaggtctagaggggcgacattttgtactgtgcttgactccacgctcggtccactagcgagtgttagtagtagcactgttgcttcgtagcggagcatgatggccgtgggagcttccccttggtaacaaggacccacggggccaaaagccacgtcctaccggacccatcatgtgtgcaaacccagcacggcaactttactgcgaaaaccactttaaggtgacactgatactggtactcaatcactggtgacaggctaaggatgcccttcaggtaccccgaggtaacacgcgacactcgggatctgagaaggggactggggcttctttaaaagtgcccagtttaaaaagcttctatgcctgaataggcgaccggaggccggcgccttttcactgttttactactgttttcatgaatacaactgactgtttcaccgccctgttacacgctctcagagagatcaaaacactgtttcttttacggacacaaggaaagatggaattcacactttacaacggtgagaagaaaaccttctactccagacccaacaaccacgacaactgctggcttaacaccattctccagttgttcaggtatgttgatgagcctttctttgactgggtctacgactcgcctgaaaacctcactcttgaggcaatcaaacagttggaagagacaaccggtcttgagctgcacgagggtggaccacccgctctcgtcatctggaacatcaaacacttgcttcacaccggaatcggcactgcctcacgccctagcgaggtgtgtatggtggacggaacggacatgtgtttagctgattttcatgctggcattttcctgaaaggacaggaacatgctgtgttcgcctgtgtcacctccaacgggtggtacgcgattgatgacgaggacttttacccttggacaccggacccgtccgacgttctggtgtttgtcccgtacgatcaagaaccgcttaacggagagtggaaaacaaaggtccagaaaaggctcaagggagccgggcaatccagcccggcaaccgggtcacagaaccaatcaggcaacactgggagcatcatcaacaattactacatgcagcagtaccaaaactccatggacacacaacttggtgacaacgctacaagcggaggctcaaacgaggggtccacggacacaacctccacccacacaaccaacactcagaacaacgactggttctcgaagctggccagttccgctttcagcggtcttttcggcgctcttctcgccgacaagaaaaccgaggagaccactcttctcgaggaccgcatcctcactactcgtaacggacacaccacctcgacaacccagtcgagcgttggagtcacgtacgggtatgcaacagctgaggatttcgtgagcgggccaaacacctctggtctcgagaccagggttgcccaggcagagcggttctttaaaacccacctgttcgactgggtcaccagtgacccattcggacggtgccacctgctggaacttccaactgaccacaaaggtgtctacggcagcctgaccgactcgtatgcttatatgaggaacggctgggatgttgaagtcactgcagtgggaaaccagttcaatggaggatgcctgttggtggccatggtgccagaactttgctccatacagaagagggagctgtaccagctcacgctctttcctcaccagttcatcaaccctcggacgaacatgacagcacacatcactgtgccctttgttggcgtcaaccgttatgaccagtacaaggtacacaaaccttggaccctcgtggttatggttgtagcccccctgactgtcaacagtgaaggtgccccgcaaatcaaggtgtatgccaacatcgcacctaccaacgtacacgtcgcgggtgagttcccttccaaagaggggatcttccctgtggcttgcagcgatggttatggcggtctggtgaccactgacccgaaaacggctgaccccgcttacgggaaagtgtttaaccccccccgcaacatgttgccggggcggttcaccaattttcttgacgtggctgaggcgtgccccacgtttctccacttcgagggtgacgtgccatacgtgaccacgaagacggattcagacagggtgctcgctcagttcgacttgtctttggcagcaaagcacatgtcgaacaccttccttgcaggtctcgcccagtactacacacagtacagcggcaccatcaacctgcacttcatgttcacagggcctactgacgcgaaggcgcgttacatgattgcgtatgctcctcctggcatggaaccacctaaaacgccagaggcggctgcccactgcattcatgctgaatgggacacagggttgaactcaaaattcacattttcaatcccttacctttcggcggctgattacgcttacacagcgtctgacactgctgagaccacaaatgtacagggatgggtttgcctgtttcaaataacacacgggaaagctgacggcgacgcactggtcgttttggctagcgccggaaaggactttgagctgcgcctgccggtggatgctcgcacacagactacctccgcgggcgagtcagctgaccccgtgaccgccaccgttgagaattacggtggcgagacacaggtccagaggcgccaacacacggacgtctcatttatattagacagatttgtgacagtgacaccaaaagaccaaattaatgtattggacctgatgcaaacccctgctcacactttggtgggagcactccttcgtactgccacttactatttcgctgacttagaggtggcagtgaagcacaagggaaacctcacctgggtcccgaacggggcgcctgaagcggcgttggacaacaccaccaacccaacagcttaccacaaggcaccactcacccgacttgcactgccttacacggcgccacaccgcgtgttggctactgtttacaacgggaactgcaagtatggtgacggcacggtggccaatgtgagaggtgacctgcaagtgttggcccagaaggcggcgagagcgctgcctacctccttcaactacggtgccattaaagctactcgggtgactgaactgctttaccgcatgaagagggctgagacatactgcccccggcctcttttggccattcacccggaccaggctagacacaagcagaagattgtggcaccggtggaacagcttctaaattttgacctgctcaaattggcgggagatgtggagtccaaccctgggcccttcttcttctccgacgtcaggtcaaatttctcaaaactggtagaaaccatcaatcagatgcaggaggacatgtcaacaaaacacgggcctgactttaaccggttggtgtccgcatttgaggaattggccactggagtgaaggctatcagggccggtctcgacgaggccaaaccctggtacaaactcatcaagctcctgagccgcttgtcgtgcatggccgctgtagcagcacggtcaaaggacccagtccttgtggccatcatgctggctgacaccggtcttgagattctggacagcacctttgtcgtgaagaagatctccgactcgctctccagtctctttcacgtgccggcccccgtcttcagtttcggagccccgattctgttggccgggttggtcaaagtcgcctcgagtttcttccggtccacacccgaagaccttgagagagcagaaaaacagctcaaagcacgtgacattaacgacatattcgccattctcaagaacggcgagtggctggtcaagctgatccttgccatccgcgactggatcaaagcgtggatcgcctcagaagaaaagtttgtcaccatgacggacttggtgcctggtatccttgaaaagcagcgggatctcaacgacccgagtaagtacaaggaagccaaggagtggctcgacaacgcgcgccaggcgtgtttgaagagcgggaacgttcacattgccaatttgtgcaaagtggtcgccccggcacccagcaagtcgagacccgaacccgtggtcgtttgcctccgcggcaaatccggccaggggaagagtttccttgcgaacgtgctcgcgcaagcaatctccacccacttcaccggcagaactgattcggtttggtactgcccgcctgaccctgaccacttcgacggttacaaccagcagaccgttgtcgtgatggacgatttgggccagaaccccgatggcaaggacttcaagtacttcgcccagatggtttcgaccacggggttcatcccgcccatggcctcgcttgaggacaaaggcaagcctttcaacagcaaagtcatcattgctaccaccaacctgtactcgggtttcaccccgagaacaatggtgtgtcctgacgcgctgaaccggaggttccactttgacatcgacgtgagtgccaaggacgggtacaaagttaacaacaaattggacataatcaaagctcttgaagacacccacaccaacccagtggcgatgttccaatacgactgtgcccttctaaacggtatggcagttgaaatgaagagaatgcaacaggatatgttcaagcctcaaccacccctccagaacgtgtaccaactcgttcacgaggtgattgaacgggtcgagctccacgagaaggtgtcgagccacccgattttcaaacagatatcaattccttcccaaaagtctgtgttgtacttcctcattgagaaaggccaacacgaagcagcaattgaattctttgagggaatggtgcatgactccatcaaggaagagctccggcccctcatccaacagacctcatttgtgaaacgcgcttttaagcgcctgaaggaaaactttgagactgttgccctgtgtttgactcttttggcaaacatagtgatcatgatccgcgagactcgcaagagacaacagatggtggacgatgcagtgaatgactacattgagaaggcaaacatcaccacagatgacaagactcttgacgaggcggaaaagaaccctctagagaccagcggtgccagcactattggtttcagagagagaactctcccggggcacaaggcgagcgatgacgtgagctccgagcccgccaaacccgtggaggaccgaccacaagctgaagggccctacgccggaccacttgagcgtcagaaacctctgagagtgaaaaccaagttgccacaacaggagggaccctacgctggcccgatggatagacagaaaccgttgaaagtgagagcaagagccccggtcgtgaaggagggaccctacgagggaccggtgaagaagcctgtcgctttgaaagtgaaagccaagaacttgattgtcactgagagtggtgccccaccgaccgacttgcagaagatggtcatgggcaacactaagcctgttgagctcatcctcgacgggaagacggtagccatctgctgtgctaccggagtgtttggcactgcctacctcgtacctcgtcacctcttcgcggagaagtacgacaagataatgttggacggtagagccatgacagacagtgactacagagtgtttgagtttgagattaaagtaaaaggacaggacatgctctcagacgctgcactcatggtgcttcaccgtgggaaccgcgtgagagacatcacgaaacattttcgtgacacagcaagaatgaagaaaggcacccccgttgtcggtgtgatcaacaacgccgacgttgggagactgattttctctggagaggcccttacctacaaagacattgtagtgtgcatggatggagacaccatgccgggcctgtttgcctacagagccgccaccaaggctggttactgcgggggagccgttctcgccaaggacggagccgacacattcatcgttggcacccactccgcaggtggtaacggagttggatactgctcgtgcgtgtccaggtccatgctcctgaaaatgaaggcacacattgaccctgaaccacaccacgaggggttgattgttgataccagagatgtggaagagcgcgtgcatgtcatgcgtaaaaccaagcttgcacccaccgtggcacacggtgtgtttaaccctgaatttggtcccgctgccttgtccaacaaggacccgcggctgaacgaaggggttgtcctcgatgaagtcatcttctccaaacacaagggagacacgaaaatgtctgaggaggacaaagcgctgttccgccgctgcgctgccgactacgcgtcgcacttgcacagcgtgctggggacggcaaatgccccattgagcatctatgaggccatcaaaggcgtcgacgggctcgatgccatggagccggacaccgcgcccggcctcccctgggccctccaggggaaacgccgtggtgcgttgattgacttcgagaacggcacggtcggacccgaagtcgaggctgccctaaagctcatggagaaaagagagtacaaatttgcttgccagaccttcctgaaagacgagattcgtccgatggaaaaagtacgtgctggcaagactcgcattgtcgacgttttgcccgtggaacacattctttacaccaggatgatgattggcagattctgtgctcaaatgcacacaaacaatggaccgcagattggctcagcggtcggttgcaatcctgatgttgattggcaaagatttggcacacattttgctcagtacagaaacgtgtgggatgtggactattcggcctttgatgctaaccactgcagtgacgcaatgaacatcatgtttgaggaggtatttcgcacagacttcggtttccacccaaatgctgagtggattctgaagactcttgtgaacacggagcacgcctatgagaacaaacgtatcactgttgagggcgggatgccgtctggctgttccgcgacaagcatcatcaacacaattttgaacaacatttatgtgctctacgctcttcgtagacactatgagggagttgagctggacacctacaccatgatctcctacggagatgacatcgtggttgcaagtgactacgatctggattttgaggctctcaaaccccacttcaaatctcttggtcaaaccatcactccagctgacaaaagcgacaaaggttttgttcttggtcactccattaccgatgtcactttcctcaaaagacacttccacatggactatggaactgggttttacaaacctgtgatggcctcaaagaccctcgaggccattctctcctttgcacgccgtgggaccatacaggagaagttgatctccgtggcaggactcgccgtccactcaggacctgacgagtaccggcgtctctttgagcccttccagggtctcttcgagattccaagctacagatcactttacctgcgttgggtgaacgccgtgtgcggtgacgcataatccctcagatgtcacaattggcagaaagactctgaggcgagcgacgccgtaggagtgaaaagcccgaaagggcttttcccgcttcctattccaaaaaaaaaaaaaaaaaactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 2 - DNA, 2202 bp, Foot-and-mouth disease virus
  source 1..2202: mol_type = genomic DNA
ggcgccgggcaatccagcccggcgactgggtcacagaaccagtcaggcaacactggaagcatcatcaacaactactacatgcagcagtaccagaactccatggacacacaacttggtgacaacgctatcagcggaggctccaacgaggggtccacggacaccacttccacccacacaaccaacactcagaacaacgactggttttcaaagctggccagttccgcttttagcggccttttcggcgctcttctcgccgacaagaaaaccgaggagaccactcttctcgaggaccgcatcctcactacccgcaacggacatacaacctcgacaacccagtcgagcgttggagtcacttacgggtacgcaacagctgaggactttgtgagcgggccaaacacatccggtcttgagaccagggttgtgcaagcagagcggttcttcaaaacccacttgttcgactgggtcactagcgacccgttcggacggtgccacctgctggaacttccaactgaccacaaaggtgtctacggcagcctgaccgattcttatgcttacatgagaaacggttgggatgttgaggtcactgcagtgggaaaccagttcaacggaggatgcctgttggtagccatggtgccagaactttgctctattgacaaaagagagctgtaccagctcacgctctttccccaccaattcatcaacccccggacgaacatgacggcgcacatcaccgtgccctttgttggcgtcaatcgctacgaccagtacaaggtacacaagccttggaccctcgtggtcatggtcgtggccccgctgactgtcaacactgaaggtgctccacagatcaaggtttatgccaacatcgcccctaccaacgtgcacgtcgcgggtgagttcccttccaaggaagggatcttccccgtggcatgtagcgacggttatggcggtcttgtgaccactgacccaaagacggctgaccccgcctacgggaaagttttcaatccccctcgcaacatgttgccagggcggttcaccaacttccttgacgtggctgaggcgtgccctacgtttctgcactttgagggtgacgtgccatacgtgaccacaaagacggattcggacagggttcttgctcagtttgacttgtctttggcagcgaagcacatgtcgaacacctttctggcaggtctcgcccagtactacacacagtacagcggcaccatcaacctgcacttcatgttcacagggcccactgacgcgaaagcgcgttacatgattgcatacgccccccctggcatggaaccgcccagaacacctgaggcggccgctcactgcattcatgcggagtgggacactgggttgaattcaaaattcacattttcaatcccttacctttcggcggctgactacgcgtacaccgcgtctgacactgctgagaccacaaatgtacagggatgggtttgcctgtttcagatcacacacgggaaggctgacggtgacgcacttgtcgttctggctagcgccggtaaggacttcgagctgcggttgccagttgacgctcgcacgcagaccacctccacaggtgagtcagctgaccccgtgactgccactgttgagaactacggtggcgagacgcaggtccagagacgccagcacacggacgtctcgttcatactggacagatttgtgaaagtgacaccaaaagaccaaattaatgtgttggacctgatgcagacccccgcccacactttggtaggtgcgcttctccgcaccgccacctactacttcgcagacctagaggtggcagtgaaacacgaggggaaccttacctgggtcccgaatggggcgcccgagacagcgttggataacaccaccaatccaacggcttaccacaaggcacctctcacccggcttgcgctgccttacacggcaccacaccgtgtcttggctactgtttacaacgggaactgcaagtatggcgagagctccacaaccaacgtgagaggtgacctgcaagtgttggcccagaaagcggcgagagcgctgcctacctcctttaactacggtgccattaaggccactcgggtgactgaactgctttaccgcatgaagagggctgaaacatactgcccccggcctcttttggctattcacccgagcgaagcaagacacaaacaaaagatagtggcacctgtgaaacag

SEQ ID NO: 3 - DNA, 11100 bp, synthetic construct
  source 1..11100: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcatatgtaatacgactcactatagggttgaaagggggcgctagggtctcacccctagcatgccaacgacagctcctacgtcgcactccacactaacgtttgtgtgcgcgcgggaaccgatggacttttgttcacccacctacagttggactcacggcaccgcgtggccattttagctgggttgtgcggacgaacactgcttgcgcatctcgcgtgaccggttagtactcttaccactatccgcctacttggtcgttagcgctgtcctgggcactcttgttgggggctgttcaacgctctacggtctcccctgcgtaacagactacggtgttggggccgcttcgtgcgagccgatcgcttggtgtgcctcggctgtcgcccgaagcccgcctttcaccccccccccccccccccccctaggttttaccgtcgttcccgacgttaatggggaaacaaccacaagcttaacaccgtcttgcccgacgtaaaagggctgcaaccaaaaagcttgtgccgcctttcccggcgttaatgggaggtaaccacaagacaaaccttcacccggaagtaaaacggcaacttcacacagttttgcccgttttcgtgagaaatgggccgtcaacgcacgaaacgcgccgtcgcttgaggaggacttgtacaaacacgatctatgcaggtttccacaactgacacaaaccgtgcaacttgaaaccccgcctggtctttccaggtctagaggggcgacattttgtactgtgcttgactccacgctcggtccactagcgagtgttagtagtagcactgttgcttcgtagcggagcatgatggccgtgggagcttccccttggtaacaaggacccacggggccaaaagccacgtcctaccggacccatcatgtgtgcaaacccagcacggcaactttactgcgaaaaccactttaaggtgacactgatactggtactcaatcactggtgacaggctaaggatgcccttcaggtaccccgaggtaacacgcgacactcgggatctgagaaggggactggggcttctttaaaagtgcccagtttaaaaagcttctatgcctgaataggcgaccggaggccggcgccttttcactgttttactactgttttcatgaatacaactgactgtttcaccgccctgttacacgctctcagagagatcaaaacactgtttcttttacggacacaaggaaagatggaattcacactttacaacggtgagaagaaaaccttctactccagacccaacaaccacgacaactgctggcttaacaccattctccagttgttcaggtatgttgatgagcctttctttgactgggtctacgactcgcctgaaaacctcactcttgaggcaatcaaacagttggaagagacaaccggtcttgagctgcacgagggtggaccacccgctctcgtcatctggaacatcaaacacttgcttcacaccggaatcggcactgcctcacgccctagcgaggtgtgtatggtggacggaacggacatgtgtttagctgattttcatgctggcattttcctgaaaggacaggaacatgctgtgttcgcctgtgtcacctccaacgggtggtacgcgattgatgacgaggacttttacccttggacaccggacccgtccgacgttctggtgtttgtcccgtacgatcaagaaccgcttaacggagagtggaaaacaaaggtccagaaaaggctcaagggcgccgggcaatccagcccggcgactgggtcacagaaccagtcaggcaacactggaagcatcatcaacaactactacatgcagcagtaccagaactccatggacacacaacttggtgacaacgctatcagcggaggctccaacgaggggtccacggacaccacttccacccacacaaccaacactcagaacaacgactggttttcaaagctggccagttccgcttttagcggccttttcggcgctcttctcgccgacaagaaaaccgaggagaccactcttctcgaggaccgcatcctcactacccgcaacggacatacaacctcgacaacccagtcgagcgttggagtcacttacgggtacgcaacagctgaggactttgtgagcgggccaaacacatccggtcttgagaccagggttgtgcaagcagagcggttcttcaaaacccacttgttcgactgggtcactagcgacccgttcggacggtgccacctgctggaacttccaactgaccacaaaggtgtctacggcagcctgaccgattcttatgcttacatgagaaacggttgggatgttgaggtcactgcagtgggaaaccagttcaacggaggatgcctgttggtagccatggtgccagaactttgctctattgacaaaagagagctgtaccagctcacgctctttccccaccaattcatcaacccccggacgaacatgacggcgcacatcaccgtgccctttgttggcgtcaatcgctacgaccagtacaaggtacacaagccttggaccctcgtggtcatggtcgtggccccgctgactgtcaacactgaaggtgctccacagatcaaggtttatgccaacatcgcccctaccaacgtgcacgtcgcgggtgagttcccttccaaggaagggatcttccccgtggcatgtagcgacggttatggcggtcttgtgaccactgacccaaagacggctgaccccgcctacgggaaagttttcaatccccctcgcaacatgttgccagggcggttcaccaacttccttgacgtggctgaggcgtgccctacgtttctgcactttgagggtgacgtgccatacgtgaccacaaagacggattcggacagggttcttgctcagtttgacttgtctttggcagcgaagcacatgtcgaacacctttctggcaggtctcgcccagtactacacacagtacagcggcaccatcaacctgcacttcatgttcacagggcccactgacgcgaaagcgcgttacatgattgcatacgccccccctggcatggaaccgcccagaacacctgaggcggccgctcactgcattcatgcggagtgggacactgggttgaattcaaaattcacattttcaatcccttacctttcggcggctgactacgcgtacaccgcgtctgacactgctgagaccacaaatgtacagggatgggtttgcctgtttcagatcacacacgggaaggctgacggtgacgcacttgtcgttctggctagcgccggtaaggacttcgagctgcggttgccagttgacgctcgcacgcagaccacctccacaggtgagtcagctgaccccgtgactgccactgttgagaactacggtggcgagacgcaggtccagagacgccagcacacggacgtctcgttcatactggacagatttgtgaaagtgacaccaaaagaccaaattaatgtgttggacctgatgcagacccccgcccacactttggtaggtgcgcttctccgcaccgccacctactacttcgcagacctagaggtggcagtgaaacacgaggggaaccttacctgggtcccgaatggggcgcccgagacagcgttggataacaccaccaatccaacggcttaccacaaggcacctctcacccggcttgcgctgccttacacggcaccacaccgtgtcttggctactgtttacaacgggaactgcaagtatggcgagagctccacaaccaacgtgagaggtgacctgcaagtgttggcccagaaagcggcgagagcgctgcctacctcctttaactacggtgccattaaggccactcgggtgactgaactgctttaccgcatgaagagggctgaaacatactgcccccggcctcttttggctattcacccgagcgaagcaagacacaaacaaaagatagtggcacctgtgaaacagcttctaaattttgacctgctcaaattggcgggagatgtggagtccaaccctgggcccttcttcttctccgacgtcaggtcaaatttctcaaaactggtagaaaccatcaatcagatgcaggaggacatgtcaacaaaacacgggcctgactttaaccggttggtgtccgcatttgaggaattggccactggagtgaaggctatcagggccggtctcgacgaggccaaaccctggtacaaactcatcaagctcctgagccgcttgtcgtgcatggccgctgtagcagcacggtcaaaggacccagtccttgtggccatcatgctggctgacaccggtcttgagattctggacagcacctttgtcgtgaagaagatctccgactcgctctccagtctctttcacgtgccggcccccgtcttcagtttcggagccccgattctgttggccgggttggtcaaagtcgcctcgagtttcttccggtccacacccgaagaccttgagagagcagaaaaacagctcaaagcacgtgacattaacgacatattcgccattctcaagaacggcgagtggctggtcaagctgatccttgccatccgcgactggatcaaagcgtggatcgcctcagaagaaaagtttgtcaccatgacggacttggtgcctggtatccttgaaaagcagcgggatctcaacgacccgagtaagtacaaggaagccaaggagtggctcgacaacgcgcgccaggcgtgtttgaagagcgggaacgttcacattgccaatttgtgcaaagtggtcgccccggcacccagcaagtcgagacccgaacccgtggtcgtttgcctccgcggcaaatccggccaggggaagagtttccttgcgaacgtgctcgcgcaagcaatctccacccacttcaccggcagaactgattcggtttggtactgcccgcctgaccctgaccacttcgacggttacaaccagcagaccgttgtcgtgatggacgatttgggccagaaccccgatggcaaggacttcaagtacttcgcccagatggtttcgaccacggggttcatcccgcccatggcctcgcttgaggacaaaggcaagcctttcaacagcaaagtcatcattgctaccaccaacctgtactcgggtttcaccccgagaacaatggtgtgtcctgacgcgctgaaccggaggttccactttgacatcgacgtgagtgccaaggacgggtacaaagttaacaacaaattggacataatcaaagctcttgaagacacccacaccaacccagtggcgatgttccaatacgactgtgcccttctaaacggtatggcagttgaaatgaagagaatgcaacaggatatgttcaagcctcaaccacccctccagaacgtgtaccaactcgttcacgaggtgattgaacgggtcgagctccacgagaaggtgtcgagccacccgattttcaaacagatatcaattccttcccaaaagtctgtgttgtacttcctcattgagaaaggccaacacgaagcagcaattgaattctttgagggaatggtgcatgactccatcaaggaagagctccggcccctcatccaacagacctcatttgtgaaacgcgcttttaagcgcctgaaggaaaactttgagactgttgccctgtgtttgactcttttggcaaacatagtgatcatgatccgcgagactcgcaagagacaacagatggtggacgatgcagtgaatgactacattgagaaggcaaacatcaccacagatgacaagactcttgacgaggcggaaaagaaccctctagagaccagcggtgccagcactattggtttcagagagagaactctcccggggcacaaggcgagcgatgacgtgagctccgagcccgccaaacccgtggaggaccgaccacaagctgaaggaccttacgagggaccggtgaagaagcctgtcgctttgaaagtgaaagctaagaacttgattgtcactgaggggccatatgaaggaccagtgaagaaacctgtcgctttgaaagtgaaagcaaaagccccgattgtcactgaaggaccctacgagggaccggtgaagaagcctgtcgctttgaaagtgaaagccaagaacttgattgtcactgagagtggtgccccaccgaccgacttgcagaagatggtcatgggcaacactaagcctgttgagctcatcctcgacgggaagacggtagccatctgctgtgctaccggagtgtttggcactgcctacctcgtacctcgtcacctcttcgcggagaagtacgacaagataatgttggacggtagagccatgacagacagtgactacagagtgtttgagtttgagattaaagtaaaaggacaggacatgctctcagacgctgcactcatggtgcttcaccgtgggaaccgcgtgagagacatcacgaaacattttcgtgacacagcaagaatgaagaaaggcacccccgttgtcggtgtgatcaacaacgccgacgttgggagactgattttctctggagaggcccttacctacaaagacattgtagtgaccatggatggagacaccatgccgggcctgtttgcctacagagccgccaccaaggctggttactgcgggggagccgttctcgccaaggacggagccgacacattcatcgttggcacccactccgcaggtggtaacggagttggatactgctcgtgcgtgtccaggtccatgctcctgaaaatgaaggcacacattgaccctgaaccacaccacgaggggttgattgttgataccagagatgtggaagagcgcgtgcatgtcatgcgtaaaaccaagcttgcacccaccgtggcacacggtgtgtttaaccctgaatttggtcccgctgccttgtccaacaaggacccgcggctgaacgaaggggttgtcctcgatgaagtcatcttctccaaacacaagggagacacgaaaatgtctgaggaggacaaagcgctgttccgccgctgcgctgccgactacgcgtcgcacttgcacagcgtgctggggacggcaaatgccccattgagcatctatgaggccatcaaaggcgtcgacgggctcgatgccatggagccggacaccgcgcccggcctcccctgggccctccaggggaaacgccgtggtgcgttgattgacttcgagaacggcacggtcggacccgaagtcgaggctgccctaaagctcatggagaaaagagagtacaaatttgcttgccagaccttcctgaaagacgagattcgtccgatggaaaaagtacgtgctggcaagactcgcattgtcgacgttttgcccgtggaacacattctttacaccaggatgatgattggcagattctgtgctcaaatgcacacaaacaatggaccgcagattggctcagcggtcggttgcaatcctgatgttgattggcaaagatttggcacacattttgctcagtacagaaacgtgtgggatgtggactattcggcctttgatgctaaccactgcagtgacgcaatgaacatcatgtttgaggaggtatttcgcacagacttcggtttccacccaaatgctgagtggattctgaagactcttgtgaacacggagcacgcctatgagaacaaacgtatcactgttgagggcgggatgccgtctggctgttccgcgacaagcatcatcaacacaattttgaacaacatttatgtgctctacgctcttcgtagacactatgagggagttgagctggacacctacaccatgatctcctacggagatgacatcgtggttgcaagtgactacgatctggattttgaggctctcaaaccccacttcaaatctcttggtcaaaccatcactccagctgacaaaagcgacaaaggttttgttcttggtcactccattaccgatgtcactttcctcaaaagacacttccacatggactatggaactgggttttacaaacctgtgatggcctcaaagaccctcgaggccattctctcctttgcacgccgtgggaccatacaggagaagttgatctccgtggcaggactcgccgtccactcaggacctgacgagtaccggcgtctctttgagcccttccagggtctcttcgagattccaagctacagatcactttacctgcgttgggtgaacgccgtgtgcggtgacgcataatccctcagatgtcacaattggcagaaagactctgaggcgagcgacgccgtaggagtgaaaagcccgaaagggcttttcccgcttcctattccaaaaaaaaaaaaaaaaaactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 4 - DNA, 39 bp, unidentified
  source 1..39: mol_type = genomic DNA
ggtaagcagctctacaacgtggaggccacatcctatgcc

SEQ ID NO: 5 - protein, 13 aa, unidentified
GKQLYNVEATSYA

SEQ ID NO: 6 - DNA, 39 bp, synthetic construct
  source 1..39: mol_type = other DNA
ggaggccacatcctatgcccgcgagaggccctaggtcgc

SEQ ID NO: 7 - DNA, 40 bp, synthetic construct
  source 1..40: mol_type = other DNA
acgttgtagagctgcttaccgcgagggtcgccgctcagct

SEQ ID NO: 8 - DNA, 11139 bp, synthetic construct
  source 1..11139: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcatatgtaatacgactcactatagggttgaaagggggcgctagggtctcacccctagcatgccaacgacagctcctacgtcgcactccacactaacgtttgtgtgcgcgcgggaaccgatggacttttgttcacccacctacagttggactcacggcaccgcgtggccattttagctgggttgtgcggacgaacactgcttgcgcatctcgcgtgaccggttagtactcttaccactatccgcctacttggtcgttagcgctgtcctgggcactcttgttgggggctgttcaacgctctacggtctcccctgcgtaacagactacggtgttggggccgcttcgtgcgagccgatcgcttggtgtgcctcggctgtcgcccgaagcccgcctttcaccccccccccccccccccccctaggttttaccgtcgttcccgacgttaatggggaaacaaccacaagcttaacaccgtcttgcccgacgtaaaagggctgcaaccaaaaagcttgtgccgcctttcccggcgttaatgggaggtaaccacaagacaaaccttcacccggaagtaaaacggcaacttcacacagttttgcccgttttcgtgagaaatgggccgtcaacgcacgaaacgcgccgtcgcttgaggaggacttgtacaaacacgatctatgcaggtttccacaactgacacaaaccgtgcaacttgaaaccccgcctggtctttccaggtctagaggggcgacattttgtactgtgcttgactccacgctcggtccactagcgagtgttagtagtagcactgttgcttcgtagcggagcatgatggccgtgggagcttccccttggtaacaaggacccacggggccaaaagccacgtcctaccggacccatcatgtgtgcaaacccagcacggcaactttactgcgaaaaccactttaaggtgacactgatactggtactcaatcactggtgacaggctaaggatgcccttcaggtaccccgaggtaacacgcgacactcgggatctgagaaggggactggggcttctttaaaagtgcccagtttaaaaagcttctatgcctgaataggcgaccggaggccggcgccttttcactgttttactactgttttcatgaatacaactgactgtttcaccgccctgttacacgctctcagagagatcaaaacactgtttcttttacggacacaaggaaagatggaattcacactttacaacggtgagaagaaaaccttctactccagacccaacaaccacgacaactgctggcttaacaccattctccagttgttcaggtatgttgatgagcctttctttgactgggtctacgactcgcctgaaaacctcactcttgaggcaatcaaacagttggaagagacaaccggtcttgagctgcacgagggtggaccacccgctctcgtcatctggaacatcaaacacttgcttcacaccggaatcggcactgcctcacgccctagcgaggtgtgtatggtggacggaacggacatgtgtttagctgattttcatgctggcattttcctgaaaggacaggaacatgctgtgttcgcctgtgtcacctccaacgggtggtacgcgattgatgacgaggacttttacccttggacaccggacccgtccgacgttctggtgtttgtcccgtacgatcaagaaccgcttaacggagagtggaaaacaaaggtccagaaaaggctcaagggcgccgggcaatccagcccggcgactgggtcacagaaccagtcaggcaacactggaagcatcatcaacaactactacatgcagcagtaccagaactccatggacacacaacttggtgacaacgctatcagcggaggctccaacgaggggtccacggacaccacttccacccacacaaccaacactcagaacaacgactggttttcaaagctggccagttccgcttttagcggccttttcggcgctcttctcgccgacaagaaaaccgaggagaccactcttctcgaggaccgcatcctcactacccgcaacggacatacaacctcgacaacccagtcgagcgttggagtcacttacgggtacgcaacagctgaggactttgtgagcgggccaaacacatccggtcttgagaccagggttgtgcaagcagagcggttcttcaaaacccacttgttcgactgggtcactagcgacccgttcggacggtgccacctgctggaacttccaactgaccacaaaggtgtctacggcagcctgaccgattcttatgcttacatgagaaacggttgggatgttgaggtcactgcagtgggaaaccagttcaacggaggatgcctgttggtagccatggtgccagaactttgctctattgacaaaagagagctgtaccagctcacgctctttccccaccaattcatcaacccccggacgaacatgacggcgcacatcaccgtgccctttgttggcgtcaatcgctacgaccagtacaaggtacacaagccttggaccctcgtggtcatggtcgtggccccgctgactgtcaacactgaaggtgctccacagatcaaggtttatgccaacatcgcccctaccaacgtgcacgtcgcgggtgagttcccttccaaggaagggatcttccccgtggcatgtagcgacggttatggcggtcttgtgaccactgacccaaagacggctgaccccgcctacgggaaagttttcaatccccctcgcaacatgttgccagggcggttcaccaacttccttgacgtggctgaggcgtgccctacgtttctgcactttgagggtgacgtgccatacgtgaccacaaagacggattcggacagggttcttgctcagtttgacttgtctttggcagcgaagcacatgtcgaacacctttctggcaggtctcgcccagtactacacacagtacagcggcaccatcaacctgcacttcatgttcacagggcccactgacgcgaaagcgcgttacatgattgcatacgccccccctggcatggaaccgcccagaacacctgaggcggccgctcactgcattcatgcggagtgggacactgggttgaattcaaaattcacattttcaatcccttacctttcggcggctgactacgcgtacaccgcgtctgacactgctgagaccacaaatgtacagggatgggtttgcctgtttcagatcacacacgggaaggctgacggtgacgcacttgtcgttctggctagcgccggtaaggacttcgagctgcggttgccagttgacgctcgcacgcagaccacctccacaggtgagtcagctgaccccgtgactgccactgttgagaactacggtggcgagacgcaggtccagagacgccagcacacggacgtctcgttcatactggacagatttgtgaaagtgacaccaaaagaccaaattaatgtgttggacctgatgcagacccccgcccacactttggtaggtgcgcttctccgcaccgccacctactacttcgcagacctagaggtggcagtgaaacacgaggggaaccttacctgggtcccgaatggggcgcccgagacagcgttggataacaccaccaatccaacggcttaccacaaggcacctctcacccggcttgcgctgccttacacggcaccacaccgtgtcttggctactgtttacaacgggaactgcaagtatggcgagagctccacaaccaacgtgagaggtgacctgcaagtgttggccggtaagcagctctacaacgtggaggccacatcctatgcccagaaagcggcgagagcgctgcctacctcctttaactacggtgccattaaggccactcgggtgactgaactgctttaccgcatgaagagggctgaaacatactgcccccggcctcttttggctattcacccgagcgaagcaagacacaaacaaaagatagtggcacctgtgaaacagcttctaaattttgacctgctcaaattggcgggagatgtggagtccaaccctgggcccttcttcttctccgacgtcaggtcaaatttctcaaaactggtagaaaccatcaatcagatgcaggaggacatgtcaacaaaacacgggcctgactttaaccggttggtgtccgcatttgaggaattggccactggagtgaaggctatcagggccggtctcgacgaggccaaaccctggtacaaactcatcaagctcctgagccgcttgtcgtgcatggccgctgtagcagcacggtcaaaggacccagtccttgtggccatcatgctggctgacaccggtcttgagattctggacagcacctttgtcgtgaagaagatctccgactcgctctccagtctctttcacgtgccggcccccgtcttcagtttcggagccccgattctgttggccgggttggtcaaagtcgcctcgagtttcttccggtccacacccgaagaccttgagagagcagaaaaacagctcaaagcacgtgacattaacgacatattcgccattctcaagaacggcgagtggctggtcaagctgatccttgccatccgcgactggatcaaagcgtggatcgcctcagaagaaaagtttgtcaccatgacggacttggtgcctggtatccttgaaaagcagcgggatctcaacgacccgagtaagtacaaggaagccaaggagtggctcgacaacgcgcgccaggcgtgtttgaagagcgggaacgttcacattgccaatttgtgcaaagtggtcgccccggcacccagcaagtcgagacccgaacccgtggtcgtttgcctccgcggcaaatccggccaggggaagagtttccttgcgaacgtgctcgcgcaagcaatctccacccacttcaccggcagaactgattcggtttggtactgcccgcctgaccctgaccacttcgacggttacaaccagcagaccgttgtcgtgatggacgatttgggccagaaccccgatggcaaggacttcaagtacttcgcccagatggtttcgaccacggggttcatcccgcccatggcctcgcttgaggacaaaggcaagcctttcaacagcaaagtcatcattgctaccaccaacctgtactcgggtttcaccccgagaacaatggtgtgtcctgacgcgctgaaccggaggttccactttgacatcgacgtgagtgccaaggacgggtacaaagttaacaacaaattggacataatcaaagctcttgaagacacccacaccaacccagtggcgatgttccaatacgactgtgcccttctaaacggtatggcagttgaaatgaagagaatgcaacaggatatgttcaagcctcaaccacccctccagaacgtgtaccaactcgttcacgaggtgattgaacgggtcgagctccacgagaaggtgtcgagccacccgattttcaaacagatatcaattccttcccaaaagtctgtgttgtacttcctcattgagaaaggccaacacgaagcagcaattgaattctttgagggaatggtgcatgactccatcaaggaagagctccggcccctcatccaacagacctcatttgtgaaacgcgcttttaagcgcctgaaggaaaactttgagactgttgccctgtgtttgactcttttggcaaacatagtgatcatgatccgcgagactcgcaagagacaacagatggtggacgatgcagtgaatgactacattgagaaggcaaacatcaccacagatgacaagactcttgacgaggcggaaaagaaccctctagagaccagcggtgccagcactattggtttcagagagagaactctcccggggcacaaggcgagcgatgacgtgagctccgagcccgccaaacccgtggaggaccgaccacaagctgaaggaccttacgagggaccggtgaagaagcctgtcgctttgaaagtgaaagctaagaacttgattgtcactgaggggccatatgaaggaccagtgaagaaacctgtcgctttgaaagtgaaagcaaaagccccgattgtcactgaaggaccctacgagggaccggtgaagaagcctgtcgctttgaaagtgaaagccaagaacttgattgtcactgagagtggtgccccaccgaccgacttgcagaagatggtcatgggcaacactaagcctgttgagctcatcctcgacgggaagacggtagccatctgctgtgctaccggagtgtttggcactgcctacctcgtacctcgtcacctcttcgcggagaagtacgacaagataatgttggacggtagagccatgacagacagtgactacagagtgtttgagtttgagattaaagtaaaaggacaggacatgctctcagacgctgcactcatggtgcttcaccgtgggaaccgcgtgagagacatcacgaaacattttcgtgacacagcaagaatgaagaaaggcacccccgttgtcggtgtgatcaacaacgccgacgttgggagactgattttctctggagaggcccttacctacaaagacattgtagtgaccatggatggagacaccatgccgggcctgtttgcctacagagccgccaccaaggctggttactgcgggggagccgttctcgccaaggacggagccgacacattcatcgttggcacccactccgcaggtggtaacggagttggatactgctcgtgcgtgtccaggtccatgctcctgaaaatgaaggcacacattgaccctgaaccacaccacgaggggttgattgttgataccagagatgtggaagagcgcgtgcatgtcatgcgtaaaaccaagcttgcacccaccgtggcacacggtgtgtttaaccctgaatttggtcccgctgccttgtccaacaaggacccgcggctgaacgaaggggttgtcctcgatgaagtcatcttctccaaacacaagggagacacgaaaatgtctgaggaggacaaagcgctgttccgccgctgcgctgccgactacgcgtcgcacttgcacagcgtgctggggacggcaaatgccccattgagcatctatgaggccatcaaaggcgtcgacgggctcgatgccatggagccggacaccgcgcccggcctcccctgggccctccaggggaaacgccgtggtgcgttgattgacttcgagaacggcacggtcggacccgaagtcgaggctgccctaaagctcatggagaaaagagagtacaaatttgcttgccagaccttcctgaaagacgagattcgtccgatggaaaaagtacgtgctggcaagactcgcattgtcgacgttttgcccgtggaacacattctttacaccaggatgatgattggcagattctgtgctcaaatgcacacaaacaatggaccgcagattggctcagcggtcggttgcaatcctgatgttgattggcaaagatttggcacacattttgctcagtacagaaacgtgtgggatgtggactattcggcctttgatgctaaccactgcagtgacgcaatgaacatcatgtttgaggaggtatttcgcacagacttcggtttccacccaaatgctgagtggattctgaagactcttgtgaacacggagcacgcctatgagaacaaacgtatcactgttgagggcgggatgccgtctggctgttccgcgacaagcatcatcaacacaattttgaacaacatttatgtgctctacgctcttcgtagacactatgagggagttgagctggacacctacaccatgatctcctacggagatgacatcgtggttgcaagtgactacgatctggattttgaggctctcaaaccccacttcaaatctcttggtcaaaccatcactccagctgacaaaagcgacaaaggttttgttcttggtcactccattaccgatgtcactttcctcaaaagacacttccacatggactatggaactgggttttacaaacctgtgatggcctcaaagaccctcgaggccattctctcctttgcacgccgtgggaccatacaggagaagttgatctccgtggcaggactcgccgtccactcaggacctgacgagtaccggcgtctctttgagcccttccagggtctcttcgagattccaagctacagatcactttacctgcgttgggtgaacgccgtgtgcggtgacgcataatccctcagatgtcacaattggcagaaagactctgaggcgagcgacgccgtaggagtgaaaagcccgaaagggcttttcccgcttcctattccaaaaaaaaaaaaaaaaaactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 9 - DNA, 2205 bp, Foot-and-mouth disease virus
  source 1..2205: mol_type = genomic DNA
ggagccgggcaatccagtccggcgactgggtcgcagaaccagtcaggcaacactgggagtattattaacaactactacatgcaacagtaccagaactccatggacacccaattaggtgacaacgctataagcggaggctccaatgagggatccacggacacaacttccacccacacaaccaacactcagaacaacgactggttttcaaagcttgccagttctgctttcagcggtcttttcggcgcccttctcgccgataagaagaccgaggagaccactctcctcgaggaccgcattctcaccacccgcaacgggcacaccacctccacaacccaatcgagtgtgggagtcacgtacgggtactccacccaggaagatcatgtttccggacctaacacatctggtttggagacgcgggtggtgcaggcagaaagatttttcaagaagcacctgtttgattggacaccggacaaagcttttgggcacttagagaagttggaacttcccactgaccacaagggagtctacggacacttggtggactcatttgcatacatgagaaatggctgggacgtggaggtgtccgctgttggcaaccagtttaacggcgggtgtctcctggtggccatggtccctgaatggaaagagttcaccccgcgtgagaagtaccagctcactttgtttccacaccagttcatcagccccagaaccaacatgactgcccacatcgtagtcccgtaccttggtgtgaacaggtacgaccagtataagaagcacaaaccctggacgctggttgtgatggtggtctcaccgctcaccaccaacactgttagtgcaggacaaatcaaggtttatgccaacattgccccgactcacgttcacgtggccggcgagctcccctcgaaagaggggattgtaccggtcgcttgttcggacgggtacggtggcttggtgacaacagacccaaaaacagctgaccctgtttatggtatggtgtacaacccccccaggacaaactaccccgggcggttcacaaacctgttggatgtggctgaggcctgccccacctttctctgtttcgacgaagggaaaccgtacgttgtgacaagaacggacgagcagcgtcttctggccaagttcgacgtctctcttgctgcaaagcacatgtcaaacacctacctttcagggatagcacagtactacgcacagtactctggtaccatcaacctgcacttcatgtttaccggctccacggattcaaaagcccgctacatggtggcgtacgttccacccggtgtggagacaccgccggacacgcctgagaaagctgcacactgcatccatgctgagtgggacacagggttgaactccaagtttactttctctatcccgtacgtgtctgccgcagattacgcgtacactgcgtctgatgtggcagaaacaacaaacgtacagggatgggtctgcatataccaaattacacacgggaaagctgaacaagacactctggttgtgtcggctagcgccggcaaggactttgagttgcgcctcccgattgacccccgctcacaaaccactaccaccggggagtctgcagaccctgtcaccaccactgttgaaaactacggcggtgagacacaagtccaacgacgtcagcacaccgacgttactttcataatggacagatttgtaaagatacaaaacttgaaccccacacatgtcattgacctcatgcaaacccaccaacacgggttggtaggtgccctgttacgtgctgctacgtactacttctctgacctggagattgtggtacgccatgacggtaacctaacctgggtacccaatggagcacccgaggcagctctgtctaacacgggcaaccccaccgcctacctcaaggcaccatttacgaggctcgcgctcccctacaccgcgccacaccgcgtgttggcaacagtgtacaacgggacgagcaagtactccgcaggtggtacgggcagacggggcgacctagggcctctcgcggcgagggtcgccgctcagcttcctgcttctttcaactttggtgcaattcaagccacgaccatccacgagctcctcgtgcgcatgaagcgtgccgaactctactgccccagaccactgttggcagtggaggtgtcgtctcaagacagacacaaacagaagatcattgcacctgcaaaacaa

SEQ ID NO: 10 - DNA, 11103 bp, synthetic construct
  source 1..11103: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcatatgtaatacgactcactatagggttgaaagggggcgctagggtctcacccctagcatgccaacgacagctcctacgtcgcactccacactaacgtttgtgtgcgcgcgggaaccgatggacttttgttcacccacctacagttggactcacggcaccgcgtggccattttagctgggttgtgcggacgaacactgcttgcgcatctcgcgtgaccggttagtactcttaccactatccgcctacttggtcgttagcgctgtcctgggcactcttgttgggggctgttcaacgctctacggtctcccctgcgtaacagactacggtgttggggccgcttcgtgcgagccgatcgcttggtgtgcctcggctgtcgcccgaagcccgcctttcaccccccccccccccccccccctaggttttaccgtcgttcccgacgttaatggggaaacaaccacaagcttaacaccgtcttgcccgacgtaaaagggctgcaaccaaaaagcttgtgccgcctttcccggcgttaatgggaggtaaccacaagacaaaccttcacccggaagtaaaacggcaacttcacacagttttgcccgttttcgtgagaaatgggccgtcaacgcacgaaacgcgccgtcgcttgaggaggacttgtacaaacacgatctatgcaggtttccacaactgacacaaaccgtgcaacttgaaaccccgcctggtctttccaggtctagaggggcgacattttgtactgtgcttgactccacgctcggtccactagcgagtgttagtagtagcactgttgcttcgtagcggagcatgatggccgtgggagcttccccttggtaacaaggacccacggggccaaaagccacgtcctaccggacccatcatgtgtgcaaacccagcacggcaactttactgcgaaaaccactttaaggtgacactgatactggtactcaatcactggtgacaggctaaggatgcccttcaggtaccccgaggtaacacgcgacactcgggatctgagaaggggactggggcttctttaaaagtgcccagtttaaaaagcttctatgcctgaataggcgaccggaggccggcgccttttcactgttttactactgttttcatgaatacaactgactgtttcaccgccctgttacacgctctcagagagatcaaaacactgtttcttttacggacacaaggaaagatggaattcacactttacaacggtgagaagaaaaccttctactccagacccaacaaccacgacaactgctggcttaacaccattctccagttgttcaggtatgttgatgagcctttctttgactgggtctacgactcgcctgaaaacctcactcttgaggcaatcaaacagttggaagagacaaccggtcttgagctgcacgagggtggaccacccgctctcgtcatctggaacatcaaacacttgcttcacaccggaatcggcactgcctcacgccctagcgaggtgtgtatggtggacggaacggacatgtgtttagctgattttcatgctggcattttcctgaaaggacaggaacatgctgtgttcgcctgtgtcacctccaacgggtggtacgcgattgatgacgaggacttttacccttggacaccggacccgtccgacgttctggtgtttgtcccgtacgatcaagaaccgcttaacggagagtggaaaacaaaggtccagaaaaggctcaagggagccgggcaatccagtccggcgactgggtcgcagaaccagtcaggcaacactgggagtattattaacaactactacatgcaacagtaccagaactccatggacacccaattaggtgacaacgctataagcggaggctccaatgagggatccacggacacaacttccacccacacaaccaacactcagaacaacgactggttttcaaagcttgccagttctgctttcagcggtcttttcggcgcccttctcgccgataagaagaccgaggagaccactctcctcgaggaccgcattctcaccacccgcaacgggcacaccacctccacaacccaatcgagtgtgggagtcacgtacgggtactccacccaggaagatcatgtttccggacctaacacatctggtttggagacgcgggtggtgcaggcagaaagatttttcaagaagcacctgtttgattggacaccggacaaagcttttgggcacttagagaagttggaacttcccactgaccacaagggagtctacggacacttggtggactcatttgcatacatgagaaatggctgggacgtggaggtgtccgctgttggcaaccagtttaacggcgggtgtctcctggtggccatggtccctgaatggaaagagttcaccccgcgtgagaagtaccagctcactttgtttccacaccagttcatcagccccagaaccaacatgactgcccacatcgtagtcccgtaccttggtgtgaacaggtacgaccagtataagaagcacaaaccctggacgctggttgtgatggtggtctcaccgctcaccaccaacactgttagtgcaggacaaatcaaggtttatgccaacattgccccgactcacgttcacgtggccggcgagctcccctcgaaagaggggattgtaccggtcgcttgttcggacgggtacggtggcttggtgacaacagacccaaaaacagctgaccctgtttatggtatggtgtacaacccccccaggacaaactaccccgggcggttcacaaacctgttggatgtggctgaggcctgccccacctttctctgtttcgacgaagggaaaccgtacgttgtgacaagaacggacgagcagcgtcttctggccaagttcgacgtctctcttgctgcaaagcacatgtcaaacacctacctttcagggatagcacagtactacgcacagtactctggtaccatcaacctgcacttcatgtttaccggctccacggattcaaaagcccgctacatggtggcgtacgttccacccggtgtggagacaccgccggacacgcctgagaaagctgcacactgcatccatgctgagtgggacacagggttgaactccaagtttactttctctatcccgtacgtgtctgccgcagattacgcgtacactgcgtctgatgtggcagaaacaacaaacgtacagggatgggtctgcatataccaaattacacacgggaaagctgaacaagacactctggttgtgtcggctagcgccggcaaggactttgagttgcgcctcccgattgacccccgctcacaaaccactaccaccggggagtctgcagaccctgtcaccaccactgttgaaaactacggcggtgagacacaagtccaacgacgtcagcacaccgacgttactttcataatggacagatttgtaaagatacaaaacttgaaccccacacatgtcattgacctcatgcaaacccaccaacacgggttggtaggtgccctgttacgtgctgctacgtactacttctctgacctggagattgtggtacgccatgacggtaacctaacctgggtacccaatggagcacccgaggcagctctgtctaacacgggcaaccccaccgcctacctcaaggcaccatttacgaggctcgcgctcccctacaccgcgccacaccgcgtgttggcaacagtgtacaacgggacgagcaagtactccgcaggtggtacgggcagacggggcgacctagggcctctcgcggcgagggtcgccgctcagcttcctgcttctttcaactttggtgcaattcaagccacgaccatccacgagctcctcgtgcgcatgaagcgtgccgaactctactgccccagaccactgttggcagtggaggtgtcgtctcaagacagacacaaacagaagatcattgcacctgcaaaacaacttctaaattttgacctgctcaaattggcgggagatgtggagtccaaccctgggcccttcttcttctccgacgtcaggtcaaatttctcaaaactggtagaaaccatcaatcagatgcaggaggacatgtcaacaaaacacgggcctgactttaaccggttggtgtccgcatttgaggaattggccactggagtgaaggctatcagggccggtctcgacgaggccaaaccctggtacaaactcatcaagctcctgagccgcttgtcgtgcatggccgctgtagcagcacggtcaaaggacccagtccttgtggccatcatgctggctgacaccggtcttgagattctggacagcacctttgtcgtgaagaagatctccgactcgctctccagtctctttcacgtgccggcccccgtcttcagtttcggagccccgattctgttggccgggttggtcaaagtcgcctcgagtttcttccggtccacacccgaagaccttgagagagcagaaaaacagctcaaagcacgtgacattaacgacatattcgccattctcaagaacggcgagtggctggtcaagctgatccttgccatccgcgactggatcaaagcgtggatcgcctcagaagaaaagtttgtcaccatgacggacttggtgcctggtatccttgaaaagcagcgggatctcaacgacccgagtaagtacaaggaagccaaggagtggctcgacaacgcgcgccaggcgtgtttgaagagcgggaacgttcacattgccaatttgtgcaaagtggtcgccccggcacccagcaagtcgagacccgaacccgtggtcgtttgcctccgcggcaaatccggccaggggaagagtttccttgcgaacgtgctcgcgcaagcaatctccacccacttcaccggcagaactgattcggtttggtactgcccgcctgaccctgaccacttcgacggttacaaccagcagaccgttgtcgtgatggacgatttgggccagaaccccgatggcaaggacttcaagtacttcgcccagatggtttcgaccacggggttcatcccgcccatggcctcgcttgaggacaaaggcaagcctttcaacagcaaagtcatcattgctaccaccaacctgtactcgggtttcaccccgagaacaatggtgtgtcctgacgcgctgaaccggaggttccactttgacatcgacgtgagtgccaaggacgggtacaaagttaacaacaaattggacataatcaaagctcttgaagacacccacaccaacccagtggcgatgttccaatacgactgtgcccttctaaacggtatggcagttgaaatgaagagaatgcaacaggatatgttcaagcctcaaccacccctccagaacgtgtaccaactcgttcacgaggtgattgaacgggtcgagctccacgagaaggtgtcgagccacccgattttcaaacagatatcaattccttcccaaaagtctgtgttgtacttcctcattgagaaaggccaacacgaagcagcaattgaattctttgagggaatggtgcatgactccatcaaggaagagctccggcccctcatccaacagacctcatttgtgaaacgcgcttttaagcgcctgaaggaaaactttgagactgttgccctgtgtttgactcttttggcaaacatagtgatcatgatccgcgagactcgcaagagacaacagatggtggacgatgcagtgaatgactacattgagaaggcaaacatcaccacagatgacaagactcttgacgaggcggaaaagaaccctctagagaccagcggtgccagcactattggtttcagagagagaactctcccggggcacaaggcgagcgatgacgtgagctccgagcccgccaaacccgtggaggaccgaccacaagctgaaggaccttacgagggaccggtgaagaagcctgtcgctttgaaagtgaaagctaagaacttgattgtcactgaggggccatatgaaggaccagtgaagaaacctgtcgctttgaaagtgaaagcaaaagccccgattgtcactgaaggaccctacgagggaccggtgaagaagcctgtcgctttgaaagtgaaagccaagaacttgattgtcactgagagtggtgccccaccgaccgacttgcagaagatggtcatgggcaacactaagcctgttgagctcatcctcgacgggaagacggtagccatctgctgtgctaccggagtgtttggcactgcctacctcgtacctcgtcacctcttcgcggagaagtacgacaagataatgttggacggtagagccatgacagacagtgactacagagtgtttgagtttgagattaaagtaaaaggacaggacatgctctcagacgctgcactcatggtgcttcaccgtgggaaccgcgtgagagacatcacgaaacattttcgtgacacagcaagaatgaagaaaggcacccccgttgtcggtgtgatcaacaacgccgacgttgggagactgattttctctggagaggcccttacctacaaagacattgtagtgaccatggatggagacaccatgccgggcctgtttgcctacagagccgccaccaaggctggttactgcgggggagccgttctcgccaaggacggagccgacacattcatcgttggcacccactccgcaggtggtaacggagttggatactgctcgtgcgtgtccaggtccatgctcctgaaaatgaaggcacacattgaccctgaaccacaccacgaggggttgattgttgataccagagatgtggaagagcgcgtgcatgtcatgcgtaaaaccaagcttgcacccaccgtggcacacggtgtgtttaaccctgaatttggtcccgctgccttgtccaacaaggacccgcggctgaacgaaggggttgtcctcgatgaagtcatcttctccaaacacaagggagacacgaaaatgtctgaggaggacaaagcgctgttccgccgctgcgctgccgactacgcgtcgcacttgcacagcgtgctggggacggcaaatgccccattgagcatctatgaggccatcaaaggcgtcgacgggctcgatgccatggagccggacaccgcgcccggcctcccctgggccctccaggggaaacgccgtggtgcgttgattgacttcgagaacggcacggtcggacccgaagtcgaggctgccctaaagctcatggagaaaagagagtacaaatttgcttgccagaccttcctgaaagacgagattcgtccgatggaaaaagtacgtgctggcaagactcgcattgtcgacgttttgcccgtggaacacattctttacaccaggatgatgattggcagattctgtgctcaaatgcacacaaacaatggaccgcagattggctcagcggtcggttgcaatcctgatgttgattggcaaagatttggcacacattttgctcagtacagaaacgtgtgggatgtggactattcggcctttgatgctaaccactgcagtgacgcaatgaacatcatgtttgaggaggtatttcgcacagacttcggtttccacccaaatgctgagtggattctgaagactcttgtgaacacggagcacgcctatgagaacaaacgtatcactgttgagggcgggatgccgtctggctgttccgcgacaagcatcatcaacacaattttgaacaacatttatgtgctctacgctcttcgtagacactatgagggagttgagctggacacctacaccatgatctcctacggagatgacatcgtggttgcaagtgactacgatctggattttgaggctctcaaaccccacttcaaatctcttggtcaaaccatcactccagctgacaaaagcgacaaaggttttgttcttggtcactccattaccgatgtcactttcctcaaaagacacttccacatggactatggaactgggttttacaaacctgtgatggcctcaaagaccctcgaggccattctctcctttgcacgccgtgggaccatacaggagaagttgatctccgtggcaggactcgccgtccactcaggacctgacgagtaccggcgtctctttgagcccttccagggtctcttcgagattccaagctacagatcactttacctgcgttgggtgaacgccgtgtgcggtgacgcataatccctcagatgtcacaattggcagaaagactctgaggcgagcgacgccgtaggagtgaaaagcccgaaagggcttttcccgcttcctattccaaaaaaaaaaaaaaaaaactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 11 - DNA, 11142 bp, synthetic construct
  source 1..11142: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcatatgtaatacgactcactatagggttgaaagggggcgctagggtctcacccctagcatgccaacgacagctcctacgtcgcactccacactaacgtttgtgtgcgcgcgggaaccgatggacttttgttcacccacctacagttggactcacggcaccgcgtggccattttagctgggttgtgcggacgaacactgcttgcgcatctcgcgtgaccggttagtactcttaccactatccgcctacttggtcgttagcgctgtcctgggcactcttgttgggggctgttcaacgctctacggtctcccctgcgtaacagactacggtgttggggccgcttcgtgcgagccgatcgcttggtgtgcctcggctgtcgcccgaagcccgcctttcaccccccccccccccccccccctaggttttaccgtcgttcccgacgttaatggggaaacaaccacaagcttaacaccgtcttgcccgacgtaaaagggctgcaaccaaaaagcttgtgccgcctttcccggcgttaatgggaggtaaccacaagacaaaccttcacccggaagtaaaacggcaacttcacacagttttgcccgttttcgtgagaaatgggccgtcaacgcacgaaacgcgccgtcgcttgaggaggacttgtacaaacacgatctatgcaggtttccacaactgacacaaaccgtgcaacttgaaaccccgcctggtctttccaggtctagaggggcgacattttgtactgtgcttgactccacgctcggtccactagcgagtgttagtagtagcactgttgcttcgtagcggagcatgatggccgtgggagcttccccttggtaacaaggacccacggggccaaaagccacgtcctaccggacccatcatgtgtgcaaacccagcacggcaactttactgcgaaaaccactttaaggtgacactgatactggtactcaatcactggtgacaggctaaggatgcccttcaggtaccccgaggtaacacgcgacactcgggatctgagaaggggactggggcttctttaaaagtgcccagtttaaaaagcttctatgcctgaataggcgaccggaggccggcgccttttcactgttttactactgttttcatgaatacaactgactgtttcaccgccctgttacacgctctcagagagatcaaaacactgtttcttttacggacacaaggaaagatggaattcacactttacaacggtgagaagaaaaccttctactccagacccaacaaccacgacaactgctggcttaacaccattctccagttgttcaggtatgttgatgagcctttctttgactgggtctacgactcgcctgaaaacctcactcttgaggcaatcaaacagttggaagagacaaccggtcttgagctgcacgagggtggaccacccgctctcgtcatctggaacatcaaacacttgcttcacaccggaatcggcactgcctcacgccctagcgaggtgtgtatggtggacggaacggacatgtgtttagctgattttcatgctggcattttcctgaaaggacaggaacatgctgtgttcgcctgtgtcacctccaacgggtggtacgcgattgatgacgaggacttttacccttggacaccggacccgtccgacgttctggtgtttgtcccgtacgatcaagaaccgcttaacggagagtggaaaacaaaggtccagaaaaggctcaagggagccgggcaatccagtccggcgactgggtcgcagaaccagtcaggcaacactgggagtattattaacaactactacatgcaacagtaccagaactccatggacacccaattaggtgacaacgctataagcggaggctccaatgagggatccacggacacaacttccacccacacaaccaacactcagaacaacgactggttttcaaagcttgccagttctgctttcagcggtcttttcggcgcccttctcgccgataagaagaccgaggagaccactctcctcgaggaccgcattctcaccacccgcaacgggcacaccacctccacaacccaatcgagtgtgggagtcacgtacgggtactccacccaggaagatcatgtttccggacctaacacatctggtttggagacgcgggtggtgcaggcagaaagatttttcaagaagcacctgtttgattggacaccggacaaagcttttgggcacttagagaagttggaacttcccactgaccacaagggagtctacggacacttggtggactcatttgcatacatgagaaatggctgggacgtggaggtgtccgctgttggcaaccagtttaacggcgggtgtctcctggtggccatggtccctgaatggaaagagttcaccccgcgtgagaagtaccagctcactttgtttccacaccagttcatcagccccagaaccaacatgactgcccacatcgtagtcccgtaccttggtgtgaacaggtacgaccagtataagaagcacaaaccctggacgctggttgtgatggtggtctcaccgctcaccaccaacactgttagtgcaggacaaatcaaggtttatgccaacattgccccgactcacgttcacgtggccggcgagctcccctcgaaagaggggattgtaccggtcgcttgttcggacgggtacggtggcttggtgacaacagacccaaaaacagctgaccctgtttatggtatggtgtacaacccccccaggacaaactaccccgggcggttcacaaacctgttggatgtggctgaggcctgccccacctttctctgtttcgacgaagggaaaccgtacgttgtgacaagaacggacgagcagcgtcttctggccaagttcgacgtctctcttgctgcaaagcacatgtcaaacacctacctttcagggatagcacagtactacgcacagtactctggtaccatcaacctgcacttcatgtttaccggctccacggattcaaaagcccgctacatggtggcgtacgttccacccggtgtggagacaccgccggacacgcctgagaaagctgcacactgcatccatgctgagtgggacacagggttgaactccaagtttactttctctatcccgtacgtgtctgccgcagattacgcgtacactgcgtctgatgtggcagaaacaacaaacgtacagggatgggtctgcatataccaaattacacacgggaaagctgaacaagacactctggttgtgtcggctagcgccggcaaggactttgagttgcgcctcccgattgacccccgctcacaaaccactaccaccggggagtctgcagaccctgtcaccaccactgttgaaaactacggcggtgagacacaagtccaacgacgtcagcacaccgacgttactttcataatggacagatttgtaaagatacaaaacttgaaccccacacatgtcattgacctcatgcaaacccaccaacacgggttggtaggtgccctgttacgtgctgctacgtactacttctctgacctggagattgtggtacgccatgacggtaacctaacctgggtacccaatggagcacccgaggcagctctgtctaacacgggcaaccccaccgcctacctcaaggcaccatttacgaggctcgcgctcccctacaccgcgccacaccgcgtgttggcaacagtgtacaacgggacgagcaagtactccgcaggtggtacgggcagacggggcgacctagggcctctcgcgggtaagcagctctacaacgtggaggccacatcctatgccgcgagggtcgccgctcagcttcctgcttctttcaactttggtgcaattcaagccacgaccatccacgagctcctcgtgcgcatgaagcgtgccgaactctactgccccagaccactgttggcagtggaggtgtcgtctcaagacagacacaaacagaagatcattgcacctgcaaaacaacttctaaattttgacctgctcaaattggcgggagatgtggagtccaaccctgggcccttcttcttctccgacgtcaggtcaaatttctcaaaactggtagaaaccatcaatcagatgcaggaggacatgtcaacaaaacacgggcctgactttaaccggttggtgtccgcatttgaggaattggccactggagtgaaggctatcagggccggtctcgacgaggccaaaccctggtacaaactcatcaagctcctgagccgcttgtcgtgcatggccgctgtagcagcacggtcaaaggacccagtccttgtggccatcatgctggctgacaccggtcttgagattctggacagcacctttgtcgtgaagaagatctccgactcgctctccagtctctttcacgtgccggcccccgtcttcagtttcggagccccgattctgttggccgggttggtcaaagtcgcctcgagtttcttccggtccacacccgaagaccttgagagagcagaaaaacagctcaaagcacgtgacattaacgacatattcgccattctcaagaacggcgagtggctggtcaagctgatccttgccatccgcgactggatcaaagcgtggatcgcctcagaagaaaagtttgtcaccatgacggacttggtgcctggtatccttgaaaagcagcgggatctcaacgacccgagtaagtacaaggaagccaaggagtggctcgacaacgcgcgccaggcgtgtttgaagagcgggaacgttcacattgccaatttgtgcaaagtggtcgccccggcacccagcaagtcgagacccgaacccgtggtcgtttgcctccgcggcaaatccggccaggggaagagtttccttgcgaacgtgctcgcgcaagcaatctccacccacttcaccggcagaactgattcggtttggtactgcccgcctgaccctgaccacttcgacggttacaaccagcagaccgttgtcgtgatggacgatttgggccagaaccccgatggcaaggacttcaagtacttcgcccagatggtttcgaccacggggttcatcccgcccatggcctcgcttgaggacaaaggcaagcctttcaacagcaaagtcatcattgctaccaccaacctgtactcgggtttcaccccgagaacaatggtgtgtcctgacgcgctgaaccggaggttccactttgacatcgacgtgagtgccaaggacgggtacaaagttaacaacaaattggacataatcaaagctcttgaagacacccacaccaacccagtggcgatgttccaatacgactgtgcccttctaaacggtatggcagttgaaatgaagagaatgcaacaggatatgttcaagcctcaaccacccctccagaacgtgtaccaactcgttcacgaggtgattgaacgggtcgagctccacgagaaggtgtcgagccacccgattttcaaacagatatcaattccttcccaaaagtctgtgttgtacttcctcattgagaaaggccaacacgaagcagcaattgaattctttgagggaatggtgcatgactccatcaaggaagagctccggcccctcatccaacagacctcatttgtgaaacgcgcttttaagcgcctgaaggaaaactttgagactgttgccctgtgtttgactcttttggcaaacatagtgatcatgatccgcgagactcgcaagagacaacagatggtggacgatgcagtgaatgactacattgagaaggcaaacatcaccacagatgacaagactcttgacgaggcggaaaagaaccctctagagaccagcggtgccagcactattggtttcagagagagaactctcccggggcacaaggcgagcgatgacgtgagctccgagcccgccaaacccgtggaggaccgaccacaagctgaaggaccttacgagggaccggtgaagaagcctgtcgctttgaaagtgaaagctaagaacttgattgtcactgaggggccatatgaaggaccagtgaagaaacctgtcgctttgaaagtgaaagcaaaagccccgattgtcactgaaggaccctacgagggaccggtgaagaagcctgtcgctttgaaagtgaaagccaagaacttgattgtcactgagagtggtgccccaccgaccgacttgcagaagatggtcatgggcaacactaagcctgttgagctcatcctcgacgggaagacggtagccatctgctgtgctaccggagtgtttggcactgcctacctcgtacctcgtcacctcttcgcggagaagtacgacaagataatgttggacggtagagccatgacagacagtgactacagagtgtttgagtttgagattaaagtaaaaggacaggacatgctctcagacgctgcactcatggtgcttcaccgtgggaaccgcgtgagagacatcacgaaacattttcgtgacacagcaagaatgaagaaaggcacccccgttgtcggtgtgatcaacaacgccgacgttgggagactgattttctctggagaggcccttacctacaaagacattgtagtgaccatggatggagacaccatgccgggcctgtttgcctacagagccgccaccaaggctggttactgcgggggagccgttctcgccaaggacggagccgacacattcatcgttggcacccactccgcaggtggtaacggagttggatactgctcgtgcgtgtccaggtccatgctcctgaaaatgaaggcacacattgaccctgaaccacaccacgaggggttgattgttgataccagagatgtggaagagcgcgtgcatgtcatgcgtaaaaccaagcttgcacccaccgtggcacacggtgtgtttaaccctgaatttggtcccgctgccttgtccaacaaggacccgcggctgaacgaaggggttgtcctcgatgaagtcatcttctccaaacacaagggagacacgaaaatgtctgaggaggacaaagcgctgttccgccgctgcgctgccgactacgcgtcgcacttgcacagcgtgctggggacggcaaatgccccattgagcatctatgaggccatcaaaggcgtcgacgggctcgatgccatggagccggacaccgcgcccggcctcccctgggccctccaggggaaacgccgtggtgcgttgattgacttcgagaacggcacggtcggacccgaagtcgaggctgccctaaagctcatggagaaaagagagtacaaatttgcttgccagaccttcctgaaagacgagattcgtccgatggaaaaagtacgtgctggcaagactcgcattgtcgacgttttgcccgtggaacacattctttacaccaggatgatgattggcagattctgtgctcaaatgcacacaaacaatggaccgcagattggctcagcggtcggttgcaatcctgatgttgattggcaaagatttggcacacattttgctcagtacagaaacgtgtgggatgtggactattcggcctttgatgctaaccactgcagtgacgcaatgaacatcatgtttgaggaggtatttcgcacagacttcggtttccacccaaatgctgagtggattctgaagactcttgtgaacacggagcacgcctatgagaacaaacgtatcactgttgagggcgggatgccgtctggctgttccgcgacaagcatcatcaacacaattttgaacaacatttatgtgctctacgctcttcgtagacactatgagggagttgagctggacacctacaccatgatctcctacggagatgacatcgtggttgcaagtgactacgatctggattttgaggctctcaaaccccacttcaaatctcttggtcaaaccatcactccagctgacaaaagcgacaaaggttttgttcttggtcactccattaccgatgtcactttcctcaaaagacacttccacatggactatggaactgggttttacaaacctgtgatggcctcaaagaccctcgaggccattctctcctttgcacgccgtgggaccatacaggagaagttgatctccgtggcaggactcgccgtccactcaggacctgacgagtaccggcgtctctttgagcccttccagggtctcttcgagattccaagctacagatcactttacctgcgttgggtgaacgccgtgtgcggtgacgcataatccctcagatgtcacaattggcagaaagactctgaggcgagcgacgccgtaggagtgaaaagcccgaaagggcttttcccgcttcctattccaaaaaaaaaaaaaaaaaactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 12 - DNA, 17 bp, synthetic construct
  source 1..17: mol_type = other DNA
agngcnggnaartttga

SEQ ID NO: 13 - DNA, 18 bp, synthetic construct
  source 1..18: mol_type = other DNA
catgtcntccatctggtt

SEQ ID NO: 14 - DNA, 20 bp, synthetic construct
  source 1..20: mol_type = other DNA
catctgctctctgggctgtg

SEQ ID NO: 15 - DNA, 20 bp, synthetic construct
  source 1..20: mol_type = other DNA
tgaggggatccaaagtccct

SEQ ID NO: 16 - DNA, 21 bp, synthetic construct
  source 1..21: mol_type = other DNA
tgcaaccaccacaattccaga

SEQ ID NO: 17 - DNA, 20 bp, synthetic construct
  source 1..20: mol_type = other DNA
ggtttcattccagccagtgc

SEQ ID NO: 18 - DNA, 21 bp, synthetic construct
  source 1..21: mol_type = other DNA
gccattcaaaggagcatggat

SEQ ID NO: 19 - DNA, 20 bp, synthetic construct
  source 1..20: mol_type = other DNA
ctgatggctttgcgctggat

SEQ ID NO: 20 - DNA, 22 bp, synthetic construct
  source 1..22: mol_type = other DNA
agccagtcttcattgttcaggt

SEQ ID NO: 21 - DNA, 21 bp, synthetic construct
  source 1..21: mol_type = other DNA
tcatctctttggggccatcag

SEQ ID NO: 22 - DNA, 20 bp, synthetic construct
  source 1..20: mol_type = other DNA
ctcgtgaaggcgggaatcat

SEQ ID NO: 23 - DNA, 20 bp, synthetic construct
  source 1..20: mol_type = other DNA
ggtgtgctccggttcaagat

SEQ ID NO: 24 - DNA, 20 bp, synthetic construct
  source 1..20: mol_type = other DNA
ccatatccagtgcggggatg

SEQ ID NO: 25 - DNA, 20 bp, synthetic construct
  source 1..20: mol_type = other DNA
aggccttggtggatcctttg

SEQ ID NO: 26 - DNA, 20 bp, synthetic construct
  source 1..20: mol_type = other DNA
tccctcattgcaaagcacaa

SEQ ID NO: 27 - DNA, 22 bp, synthetic construct
  source 1..22: mol_type = other DNA
gcatctcctcttgcaagcaaat

SEQ ID NO: 28 - DNA, 20 bp, synthetic construct
  source 1..20: mol_type = other DNA
aagctctggagggagtgcta

SEQ ID NO: 29 - DNA, 23 bp, synthetic construct
  source 1..23: mol_type = other DNA
caacagcagttactgtctcatca

SEQ ID NO: 30 - DNA, 20 bp, synthetic construct
  source 1..20: mol_type = other DNA
cggcccagtgaagagtttct

SEQ ID NO: 31 - DNA, 20 bp, synthetic construct
  source 1..20: mol_type = other DNA
tgccttcggcattacgtctt

SEQ ID NO: 32 - DNA, 20 bp, synthetic construct
  source 1..20: mol_type = other DNA
ggctgtcctttgatgtcacc

SEQ ID NO: 33 - DNA, 18 bp, synthetic construct
  source 1..18: mol_type = other DNA
ggccagaattgaacccgt

SEQ ID NO: 34 - DNA, 20 bp, synthetic construct
  source 1..20: mol_type = other DNA
ctcacctcccaactgatccc

SEQ ID NO: 35 - DNA, 20 bp, synthetic construct
  source 1..20: mol_type = other DNA
tgtgtccgtggacgaagttg

SEQ ID NO: 36 - DNA, 20 bp, synthetic construct
  source 1..20: mol_type = other DNA
ctgcagtcacagaacgagtg

SEQ ID NO: 37 - DNA, 20 bp, synthetic construct
  source 1..20: mol_type = other DNA
cggcatcaatctcaggtgcc

SEQ ID NO: 38 - DNA, 20 bp, synthetic construct
  source 1..20: mol_type = other DNA
gtcatcagcacaaatactgc

SEQ ID NO: 39 - DNA, 20 bp, synthetic construct
  source 1..20: mol_type = other DNA
cacaagtggtgtctgttttc

SEQ ID NO: 40 - DNA, 20 bp, synthetic construct
  source 1..20: mol_type = other DNA
tcaggcatcgttcaggtgac

SEQ ID NO: 41 - DNA, 20 bp, synthetic construct
  source 1..20: mol_type = other DNA
tgacagccagcaccatttca

SEQ ID NO: 42 - DNA, 25 bp, synthetic construct
  source 1..25: mol_type = other DNA
tgggactgagtaacattctctttgt

SEQ ID NO: 43 - DNA, 20 bp, synthetic construct
  source 1..20: mol_type = other DNA
ccagctcatccaggcttagg

SEQ ID NO: 44 - DNA, 21 bp, synthetic construct
  source 1..21: mol_type = other DNA
tgagctatttctacaccgccg

SEQ ID NO: 45 - DNA, 19 bp, synthetic construct
  source 1..19: mol_type = other DNA
tcgtccacgtagccgactt

SEQ ID NO: 46 - DNA, 20 bp, synthetic construct
  source 1..20: mol_type = other DNA
ctccagtgatgctgggtcag

SEQ ID NO: 47 - DNA, 20 bp, synthetic construct
  source 1..20: mol_type = other DNA
tgacagagtgcccgttcttc

SEQ ID NO: 48 - DNA, 20 bp, synthetic construct
  source 1..20: mol_type = other DNA
tgccatgcctacaaagctga

SEQ ID NO: 49 - DNA, 20 bp, synthetic construct
  source 1..20: mol_type = other DNA
gtagtaaccagggcggcatt

SEQ ID NO: 50 - DNA, 20 bp, synthetic construct
  source 1..20: mol_type = other DNA
tcaaaggagttccgggcatc

SEQ ID NO: 51 - DNA, 20 bp, synthetic construct
  source 1..20: mol_type = other DNA
ctgaagcaggcgggagtaat

SEQ ID NO: 52 - DNA, 20 bp, synthetic construct
  source 1..20: mol_type = other DNA
ggatgtgcagcctttgttgt

SEQ ID NO: 53 - DNA, 20 bp, synthetic construct
  source 1..20: mol_type = other DNA
cagagcgtaccaaattgcgg

SEQ ID NO: 54 - DNA, 20 bp, synthetic construct
  source 1..20: mol_type = other DNA
gagtatgggtctgcaggcaa

SEQ ID NO: 55 - DNA, 20 bp, synthetic construct
  source 1..20: mol_type = other DNA
atatgtcgcggcacagactt

SEQ ID NO: 56 - DNA, 20 bp, synthetic construct
  source 1..20: mol_type = other DNA
caccatcaccgtgactcgaa

SEQ ID NO: 57 - DNA, 20 bp, synthetic construct
  source 1..20: mol_type = other DNA
agttcgtgccgtggaatctt

SEQ ID NO: 58 - DNA, 20 bp, synthetic construct
  source 1..20: mol_type = other DNA
cccagcgtcgtgattagtga

SEQ ID NO: 59 - DNA, 20 bp, synthetic construct
  source 1..20: mol_type = other DNA
gccgttcagtcctgtccata

(19) United States
(12) Patent Application Publication
(10) Pub. No.: US 2025/0352635 A1
(43) Pub. Date: Nov. 20, 2025

(54) RECOMBINANT FOOT-AND-MOUTH DISEASE VIRUS TYPE O FOR INDUCING ROBUST ADAPTIVE IMMUNE RESPONSE AND OVERCOMING MATERNALLY-DERIVED ANTIBODY INTERFERENCE, AND FOOT-AND-MOUTH DISEASE VACCINE COMPOSITION COMPRISING SAME

(71) Applicant: REPUBLIC OF KOREA(ANIMAL AND PLANT QUARANTINE AGENCY), Gimcheon-si, Gyeongsangbuk-do (KR)

(72) Inventors: Min Ja LEE, Gimcheon-si, Gyeongsangbuk-do (KR); Hyun Mi KIM, Gimcheon-si, Gyeongsangbuk-do (KR); Sehee SHIN, Gimcheon-si, Gyeongsangbuk-do (KR); Su-Mi KIM, Daejeon (KR); Jong-Hyeon PARK, Gimcheon-si, Gyeongsangbuk-do (KR)

(21) Appl. No.: 18/710,537

(22) PCT Filed: Nov. 11, 2022
(86) PCT No.: PCT/KR2022/017801

§ 371 Date: May 15, 2024

(30) Foreign Application Priority Data
Nov. 17, 2021 (KR) 10-2021-0158591
Nov. 17, 2021 (KR) 10-2021-0158592

(51) Int. Cl. A61K 39/135; A61K 39/00; A61P 31/14; C12N 7/00; C12N 15/85; G01N 33/569

(52) U.S. Cl. CPC A61K 39/135; A61P 31/14; C12N 7/00; C12N 15/85; G01N 33/56983; A61K 2039/572; A61K 2039/575; C12N 2770/32134; G01N 2333/09

(57) ABSTRACT

Proposed are a foot-and-mouth disease vaccine composition including recombinant foot-and-mouth disease viruses and an antigen isolated and purified from the viruses. In the early stages of vaccination, a humoral immune response is simultaneously induced through the induction of a robust cellular immune response, while in the presence of maternally-derived antibodies (MDAs), B cell receptors are stimulated. Through this, it is possible to provide the vaccine composition that enables active immunity and overcomes the interference of the maternally-derived antibodies and to provide a method of preventing or treating foot-and-mouth diseases using the same composition.

### SEQUENCE LISTING

The instant application contains a Sequence Listing which has been submitted electronically in XML format and is hereby incorporated by reference in its entirety. Said Sequence Listing XML, created on Nov. 3, 2022, is named “Sequencelist_PCTKR2022017801.xml” and is 112,950 bytes in size. The sequence listing contained in this .XML file is part of the specification and is hereby incorporated by reference herein in its entirety.

### TECHNICAL FIELD

The present disclosure, to overcome the interference of maternally-derived antibodies by inducing a strong adaptive (cellular and humoral) immune response, relates to an immune-boosting recombinant foot-and-mouth disease virus with the ‘C3d gene (B cell epitope)’ inserted into a foot-and-mouth disease type O vaccine strain O1 Manisa-O PanAsia2 (O1 M-O PA2), a method of isolating and purifying inactivated antigens of the foot-and-mouth disease virus with increased immunogenicity, and use of a foot-and-mouth disease vaccine composition for overcoming interference of maternally-derived antibodies (MDAs).

### BACKGROUND ART

Foot-and-mouth disease (FMD) vaccination is required to be regular and repeated in both cattle and pigs. This vaccination induces the production of antibodies in the mother thereof. These antibodies are transferred to calves or piglets in the form of maternally-derived antibodies through the placenta or ingestion of colostrum, inducing the formation of passive immunity. These maternally-derived antibodies show host defense effects in calves and piglets during early foot-and-mouth disease virus infection. However, the MDAs have a short-term protective effect and when young animals are inoculated with foot-and-mouth disease vaccine early, the maternally-derived antibodies cause interference by passive immunity (inhibiting antigen-specific antibody production from plasma cells and memory B cells, resulting in an immunological tolerance mechanism). Therefore, the maternally-derived antibodies have negative effects of inhibiting the efficacy of vaccines and suppressing the formation of active immunity. Current foot-and-mouth disease vaccination programs recommend that calves and piglets be vaccinated after the age of 2 months when maternally-derived antibody levels decrease.

Because the level, titer, and half-life of maternally-derived antibodies vary depending on the individual, it is difficult to determine the appropriate timing for foot-and-mouth disease vaccination in the field. In addition, commercially used foot-and-mouth disease vaccines generally have a limitation in that the current vaccines are difficult to overcome the interference of maternally-derived antibodies, thereby the maternally-derived antibodies inhibit the formation of active immunity through vaccination.

Meanwhile, foot-and-mouth disease viruses (FMD viruses or FMDVs) belong to the Aphthovirus genus (Family: Picornaviridae) and are classified into seven serotypes: O, A, C, Asia1, SAT1, SAT2, and SAT3. Viruses that share more than 85% nucleotide identity to the FMDVs' genome region corresponding to the VP1 protein are mono-serotype viruses. These viruses are generally found in geographically limited regions and are classified by topotype. The FMDVs show high genetic and antigenic variation, so antibodies induced by one serotype cannot neutralize other serotypes, so there is no cross-protection upon vaccination. Nevertheless, vaccination is widely used to prevent and control the disease in countries prone to foot-and-mouth disease.

B cell activation pathways are broadly divided into three categories: 1) T cell-dependent pathway, 2) T cell-independent pathway type I, and 3) T cell-independent pathway type II. Among these, the T cell-dependent pathway is a typical pathway in which B cells are activated through TCR/MHC and CD40L/CD40. The T cell-independent pathway type I is known to be a rare pathway in the host where pathogen-associated molecular patterns (PAMPs) stimulate pattern-recognition receptors (PRRs) to directly activate B cells. Lastly, the T cell-independent pathway type II is a pathway that activates B cells by stimulating the B cell receptors CD21, CD19, and CD81 with antigens or B cell epitopes such as C3d. When maternally-derived antibodies are present in the host, immune tolerance and tolerance mechanisms occur. As a result, presenting antigens to T cells, inducing cellular immune responses, and activating B cells through T cell-dependent pathways are difficult. Therefore, it is necessary to directly activate B cells through a T cell-independent pathway or continuously stimulate T cells by inducing a robust cellular immune response.

Therefore, the present disclosure seeks to overcome the interference phenomenon of the maternally-derived antibodies, which has been pointed out as a major limitation of foot-and-mouth disease vaccines currently available on the market. To overcome the interference of the maternally-derived antibodies by stimulating receptors on the surface of B cells through C3d, which is a B cell epitope, the active site of C3d was selected as a candidate substance and inserted into the O PA2 P1 backbone (VP1 site). Thus, a foot-and-mouth disease vaccine strain of FMDV type O for overcoming interference of the maternally-derived antibodies was developed. In addition, immune-boosting antigens isolated and purified from this vaccine strain and a foot-and-mouth disease vaccine composition including the same for overcoming interference of the maternally-derived antibodies were developed.

### REFERENCES

### Patent Document

Korean Registered Patent Gazette No. 10-2234754

### Non-Patent Document

Lee, S. Y. et al. Rapid engineering of foot-and-mouth disease vaccine and challenge viruses. J. Virol. 91, e00155-00117 (2017).
Lee, M. J. et al. Advanced foot-and-mouth disease vaccine platform for stimulation of simultaneous cellular and humoral immune responses. Vaccines (Basel) 8, 254 (2020).

### SUMMARY OF INVENTION

### Technical Problem

Against the background, to overcome the interference phenomenon of maternally-derived antibodies, which is a limitation of the foot-and-mouth disease vaccines currently available on the market, the present disclosure is focused on directly stimulating receptors on the surface of B cells through C3d, which is a B cell epitope to overcome the interference of the maternally-derived antibodies.

To overcome the difficulty in inducing foot-and-mouth disease vaccine-mediated immune responses due to the interference phenomenon of the maternally-derived antibodies, which has been pointed out as a limitation of the foot-and-mouth disease vaccines currently available on the market, the objective of the present disclosure is to provide an immune-boosting recombinant foot-and-mouth disease virus with the ‘C3d gene (B cell epitope)’ inserted into the foot-and-mouth disease type O vaccine strain O1 Manisa-O PA2-R (O1 M-O PA2, hereinafter referred to as ‘O PA2’), a foot-and-mouth disease vaccine composition including antigens isolated and purified from the virus, and a method of producing the recombinant foot-and-mouth disease virus.

### Technical Solution

To achieve the objective, the present disclosure is to provide recombinant foot-and-mouth disease viruses and a foot-and-mouth disease vaccine composition including antigens isolated and purified from the recombinant foot-and-mouth disease viruses.

In addition, the present disclosure is to provide a method of producing the recombinant foot-and-mouth disease viruses and a method of isolating and purifying antigens from the recombinant foot-and-mouth disease viruses.

According to the present disclosure, the recombinant foot-and-mouth disease viruses can be produced using a recombinant plasmid into which foot-and-mouth disease virus genes are inserted. The recombinant foot-and-mouth disease viruses may be but are not limited to, foot-and-mouth disease virus type O or type A.

The recombinant foot-and-mouth disease virus type O can be produced using a recombinant plasmid represented by SEQ ID NO: 8. The recombinant foot-and-mouth disease virus type A can be produced using a recombinant plasmid represented by SEQ ID NO: 11.

To produce the recombinant foot-and-mouth disease viruses, an active site (13 amino acids) of C3d was selected as a candidate substance to be inserted into a backbone. The active site of C3d is represented by SEQ ID NO: 4 (the base sequence encoding the active site of C3d is represented by SEQ ID NO: 5).

By inserting the sequence of the active site of C3d into O PA2 or A22 P1 backbone (VP1 site), a foot-and-mouth disease vaccine composition such as O PA2-C3d of FMDV type O and A22-C3d of FMDV type A is provided to overcome the interference of maternally-derived antibodies.

In addition, the present disclosure is to provide immune-boosting antigens isolated and purified from the recombinant viruses and a foot-and-mouth disease vaccine composition including the antigens for overcoming the interference of maternally-derived antibodies.

In addition, the present disclosure is to provide a method of preventing or treating foot-and-mouth disease by using the vaccine composition including the recombinant foot-and-mouth disease viruses or antigens isolated and purified from the recombinant foot-and-mouth disease viruses.

In addition, the present disclosure is to provide a foot-and-mouth disease diagnostic kit or foot-and-mouth disease diagnostic kit composition including the recombinant foot-and-mouth disease viruses or the antigens of the recombinant foot-and-mouth disease viruses.

In addition, the present disclosure is to provide a method of diagnosing foot-and-mouth disease by using the foot-and-mouth disease diagnostic kit or foot-and-mouth disease diagnostic kit composition.

The recombinant foot-and-mouth disease viruses of the present disclosure are based on virus type O or virus type A.

The virus type O is not limited but may be O1-Manisa in one embodiment of the present disclosure.

The virus type A is not limited but may be subtype A22 in one embodiment of the present disclosure, and preferably may be A22/Iraq/24/64.

In the present disclosure, the term ‘plasmid’ refers to a DNA molecule containing a DNA sequence linked to be operable to suitable regulatory sequences capable of expressing the DNA in a suitable host. Once transformed into a suitable host, the plasmid can replicate and function independently of the host genome, or in some cases can be integrated into the genome itself. Since a plasmid is currently the most commonly used form of a vector, ‘plasmid’ and ‘vector’ are sometimes used interchangeably in the context of the present disclosure.

For the objective of the present disclosure, it is preferred to use a plasmid vector. Typical plasmid vectors that can be used for this objective have the following sites in their structures: (a) a replication origin that allows efficient replication to include hundreds of plasmid vectors per host cell, (b) a selection marker that allows host cells transformed with plasmid vectors to be selected, and (c) a restriction enzyme cutting site where foreign DNA fragments can be inserted. Even when an appropriate restriction enzyme cutting site does not exist, vectors and foreign DNAs can be easily ligated using synthetic oligonucleotide adapters or linkers according to conventional methods.

The recombinant vector and recombinant foot-and-mouth disease viruses of the present disclosure can be produced by conventional genetic manipulation and transformation methods. Appropriate amounts of viruses can be obtained by continuously subculturing viruses made in small quantities.

The cells may be derived from one or more types of cells selected from the group consisting of canines, felines, boars, bovines, deer, giraffes, peccaries, camelids, hippopotamuses, equines, tapirs, rhinoceroses, weasels, leporids, rodents, and primates. Preferably, the cells for use may be derived from one or more types selected from the group consisting of goat tongue cells (ZZ-R) and hamster kidney cells (BHK-21), black goat kidney cells (BGK), porcine kidney cells (IBRS-2), and bovine kidney cells (LFBK).

The foot-and-mouth disease vaccine composition of the present disclosure includes the recombinant foot-and-mouth disease viruses of the present disclosure or antigens isolated and purified from the recombinant viruses as an active ingredient.

The recombinant foot-and-mouth disease viruses included in the foot-and-mouth disease vaccine composition, foot-and-mouth disease diagnostic kit, and foot-and-mouth disease diagnostic kit composition of the present disclosure may be a recombinant foot-and-mouth disease virus type O or type A, respectively, or a combination thereof.

In addition, the antigens isolated and purified from the recombinant foot-and-mouth disease viruses included in the foot-and-mouth disease vaccine composition, foot-and-mouth disease diagnostic kit, and foot-and-mouth disease diagnostic kit composition of the present disclosure may be ones derived from a recombinant foot-and-mouth disease virus type O or type A, respectively, or a combination thereof.

A vaccine including the vaccine composition may be a live vaccine, an attenuated vaccine, or a killed vaccine.

The vaccine composition including the recombinant foot-and-mouth disease viruses or antigens isolated and purified from the recombinant viruses can be administered at a dose in a range of 1/640 to 1/10 dose, preferably 1/40 to 1/10 dose.

The vaccine composition can be administered to artiodactyls such as pigs, sheep, goats, deer, and wild ruminants, excluding humans.

In addition, the vaccine composition may additionally include diluents or excipients such as carriers, fillers, extenders, binders, wetting agents, disintegrants, and surfactants commonly acceptable in the art.

Additionally, the vaccine composition may be administered (or injected) to the individual in various forms. Administration may be performed by any one method selected from the group consisting of subcutaneous injection, intramuscular injection, subcutaneous injection, intraperitoneal injection, nasal administration, oral administration, transdermal administration, or oral administration.

### Advantageous Effects

The present disclosure relates to recombinant foot-and-mouth disease viruses and a foot-and-mouth disease vaccine composition including antigens isolated and purified from the recombinant foot-and-mouth disease viruses. The present disclosure can provide a vaccine composition that overcomes the interference of MDAs and enables active immunity by simultaneously inducing a humoral immune response through the induction of a robust cellular immune response in the early stage of vaccination and stimulating B cell receptors in the presence of MDAs.

### BRIEF DESCRIPTION OF DRAWINGS

FIG. 1a shows a genetic schematic diagram of a recombinant immune-boosting foot-and-mouth disease virus type O according to the present disclosure;

FIG. 1b shows a genetic schematic diagram of a recombinant immune-boosting foot-and-mouth disease virus type A according to the present disclosure;

FIGS. 2a and 2b show an immunogenicity evaluation strategy and results of antigens isolated and purified from O PA2 and A22, respectively;

FIG. 3 shows an immunogenicity evaluation strategy and results when the antigens isolated and purified from O PA2 and A22 are administered in combination;

FIG. 4a shows an experimental strategy (A), survival rate (B), and body weight change (C) for the recombinant foot-and-mouth disease virus type O according to the present disclosure;

FIG. 4b shows an experimental strategy (D), survival rate (E), and body weight change (F) for the recombinant foot-and-mouth disease virus type A according to the present disclosure;

FIG. 5 shows the results of an evaluation on vaccination and immune response induction when vaccines include the antigens isolated and purified from the recombinant immune-boosting foot-and-mouth disease viruses type O and type A are administered to mice according to the present disclosure;

FIG. 6a shows a vaccination strategy when vaccines include the antigens isolated and purified from the recombinant immune-boosting foot-and-mouth disease viruses type O and type A are administered to pigs according to the present disclosure;

FIG. 6b shows the results of evaluation on early, mid-term, and long-term immunity (antibody titer induction by SP O ELISA and SP A ELISA) in pigs depending on vaccination when vaccines include the antigens isolated and purified from the recombinant immune-boosting foot-and-mouth disease viruses type O and type A according to the present disclosure;

FIG. 7 shows the results of evaluation on early, mid-term, and long-term immunity (neutralizing antibody titer induction) in pigs depending on vaccination when vaccines include the antigens isolated and purified from the recombinant immune-boosting foot-and-mouth disease viruses type O and type A according to the present disclosure;

FIGS. 8a to 8c show the results of evaluation on the expression of cellular immune response genes (cytokines and costimulatory molecules) in pigs depending on vaccination when vaccines include the antigens isolated and purified from the recombinant immune-boosting foot-and-mouth disease viruses type O and type A according to the present disclosure;

FIG. 8d shows the results of evaluation on the induction of humoral immune responses (immunoglobulin subtypes such as IgG, IgM, and IgA) mediated by vaccination in pigs when vaccines include the antigens isolated and purified from the recombinant immune-boosting foot-and-mouth disease viruses type O and type A according to the present disclosure;

FIG. 8e shows the results of cellular immune responses in murine peritoneal exudate cells (PECs) and porcine peripheral blood mononuclear cells (PBMCs) induced by treatment of the antigens isolated and purified from the recombinant immune-boosting foot-and-mouth disease viruses type O and type A according to the present disclosure;

FIG. 9 shows the results of measuring an antigen amount in a simple kit including the antigens isolated and purified from the recombinant foot-and-mouth disease viruses according to the present disclosure and of differentiating vaccine strains of the present disclosure from wild strains;

FIG. 10 shows the results of electron microscopy (TEM) observation of the antigens (146s particle) purified using the recombinant foot-and-mouth disease viruses according to the present disclosure; and

FIG. 11 shows the VP1 sequences of O PA2-C3d and A22-C3d of the present disclosure, during the first (Sus. 1) and fourth (Sus. 4) suspension cell (BHK) passages, in which the VP1 sequences of O PA2-C3d and A22-C3d were aligned using SnapGene; (a) O PA-C3d nucleotide sequence; (b) A22-C3d nucleotide sequence; (c) O PA2-C3d amino acid sequence; and (d) A22-C3d amino acid sequence.

### MODE FOR CARRYING OUT INVENTION

Hereinafter, the present disclosure will be described in detail through examples and experimental examples.

However, the following examples and experimental examples only illustrate the present disclosure and the content of the present disclosure is not limited to the following examples and experimental examples.

### Materials and Methods

### 1. Recombinant Plasmid Preparation

A recombinant plasmid was prepared as described by Lee et al. (Lee, S. Y. et al. Rapid engineering of foot-and-mouth disease vaccine and challenge viruses. J. Virol. 91, e00155-00117 (2017).) The entire FMD-O1-Manisa virus genome (GenBank Accession No. AY593823.1) was amplified by PCR.

### (1) Construction of Recombinant Plasmid for Manufacturing Recombinant Foot-and-Mouth Disease Virus Type O

An amplified O1-Manisa genome (SEQ ID NO: 1) was inserted into a plasmid (pBluescript SK II) to prepare a pO1-Manisa (pO1 M) plasmid. In the prepared pO1 M, the gene encoding the P1 structural protein was replaced with the gene encoding the structural protein of O-serotype FMDV O PA2 (SEQ ID NO: 2) (GenBank Accession No. GU384682.1) to prepare pO1 M-O PA2 P1 (SEQ ID NO: 3) plasmid.

By using the plasmid (pO1 M-O PA2 P1) prepared as above, a B cell epitope sequence (C3d sequence (GGTAAGCAGCTCTACAACGTGGAGGCCACATCCTATGCC, SEQ ID NO. 4) corresponding to an amino acid residue sequence (GKQLYNVEATSYA, SEQ ID NO: 5) was inserted into a VP1 sequence [PA2-C3d: base pair positions 456 and 457 (amino acid positions 152 and 153, i.e., bases 2025 and 2026 of the pO1 M-O PA2 P1 sequence)]. Afterward, 300 ng/μL of pO1 M-A22 P1 was used as the PCR template addition to the use of 1 μL of 10 μmole/μL primer C3d F (5′-GGAGGCCACATCCTATGCCCGCGAGAGGCCCTAGGTCGC-3′, SEQ ID NO:6) and 1 μL of 10 μmole/μL primer C3d R (5′-ACGTTGTAGAGCTGCTTACCGCGA-GGGTCGCCGCTCAGCT-3′, SEQ ID NO: 7). The materials were used by the same self-ligating method used in previous studies to prepare a targeting plasmid. The finally prepared recombinant plasmid is the one represented by SEQ ID NO: 8.

### (2) Construction of Recombinant Plasmid for Manufacturing Recombinant Foot-and-Mouth Disease Virus Type A

An amplified O1-Manisa genome (SEQ ID NO: 1) was inserted into a plasmid (pBluescript SK II) to prepare a pO-Manisa (pO1 M) plasmid. In the prepared pO1 M, the gene encoding the structural protein was replaced with the gene encoding the structural protein of A-serotype FMDV A22/Iraq/24/64 (SEQ ID NO: 9) (GenBank Accession No. AY593764.1) to prepare O1 M-A22 P1 (SEQ ID NO: 10) plasmid.

By using the plasmid (O1 M-A22 P1) prepared as above, a B cell epitope sequence (C3d sequence (GGTAAGCAGCTCTACAACGTGGAGGCCACATCCTATGCC, SEQ ID NO. 4) corresponding to an amino acid residue sequence (GKQLYNVEATSYA, SEQ ID NO: 5) was inserted into a VP1 sequence [PA2-C3d: base pair positions 453 and 454 (amino acid positions 151 and 152, i.e., bases 2025 and 2026 of the pO1 M-O PA2 P1 sequence)]. Afterward, 300 ng/μL of O1 M-A22 P1 was used as the PCR template in addition to the use of 1 μL of 10 μmole/μL primer C3d F (5′-GGAGGCCACATCCTATGCCCGCGAGAGGCCCTAGGTCGC-3′, SEQ ID NO: 6), 1 μL of 10 μmole/μL primer C3d R (5′-ACGTTGTAGAGCTGCTTACCGCGA-GGGTCGCCGCTCAGCT-3′, SEQ ID NO: 7). The materials were used by the same self-ligating method used in previous studies to prepare a targeting plasmid. The finally prepared recombinant plasmid is the one represented by SEQ ID NO: 11.

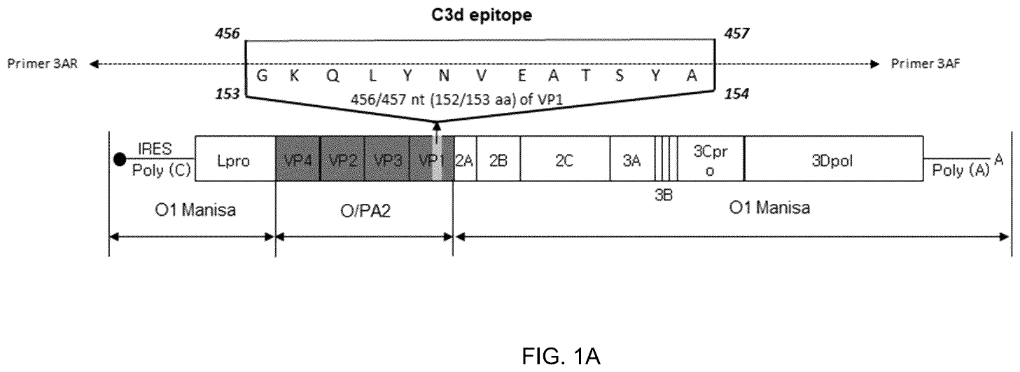

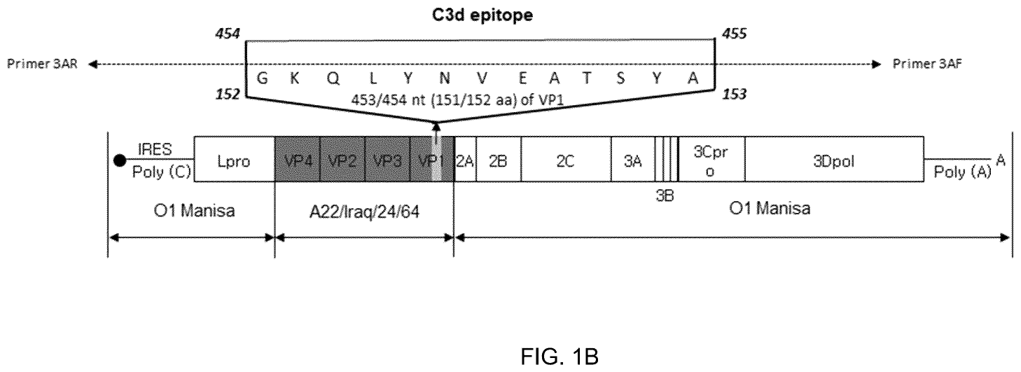

FIGS. 1a and 1b show schematic diagrams of the final plasmids for O PA2-C3d and A22-C3d, respectively.

PCR conditions were as follows: 10 μL of 5× Phusion HF buffer (Thermo Scientific, Waltham, MA, USA), 1 μL of 10 mM dNTPs (Invitrogen, Carlsbad, CA, USA), 1 μL of 2 U/μL Phusion DNA polymerase (Thermo Scientific), and 35 μL of sterile distilled water were mixed. PCR amplification with the mixture was performed at a temperature of 98° C. for 30 seconds, 98° C. for 10 seconds, 65° C. for 20 seconds, and 72° C. for 2 minutes and 30 seconds, with a final cycle of amplification at 72° C. for 10 minutes. That way, a total of 25 cycles of PCR amplification were performed. Next, 1 μL of DpnI (Enzynomics, Daejeon, Korea) was added to 25 μL of PCR product, and the mixture was reacted in an incubator at a temperature of 37° C. for 1 hour. Then, 35 μL of sterile distilled water, 5 μL of Ligation High (TOYOBO, Osaka, Japan), and 1 μL of 5 U/μL T4 polynucleotide kinase (TOYOBO, Osaka, Japan) were added to 4 μL of DpnI-treated product. The mixture was ligated in a water bath at a temperature of 16° C. for 1 hour.

After ligation, the plasmid was transformed into 100 μL of DH5 α cells (Yeast Biotech, Taipei, Taiwan) according to the manufacturer's protocol. The transformed cells were plated on agar plates containing ampicillin and cultured at a temperature of 37° C. overnight.

Colonies were picked from the plate with a pipette tip, and these colonies were mixed with 18 μL of sterile distilled water, 1 μL of 10 μmol forward universal primer VP1 (5′-AGNGCNGGNAARTTTGA-3′) (SEQ ID NO: 12), and 1 μL of 10 μmol/μL reverse universal primer. Primer VP1 (5′-CATGTCNTCCATCTGGTT-3′) (SEQ ID NO: 13) was added to the colony PCR tube, and PCR amplification was performed at a temperature of 94° C. for 5 minutes, 94° C. for 30 seconds, 55° C. for 30 seconds, 72° C. for 1 minute, with a final cycle of amplification at 72° C. for 5 minutes. That way, a total of 25 cycles of PCR amplification were performed. In the universal primer, N may represent any nucleotide. 5 μL of PCR sample was mixed with 1 μL of 6× loading buffer (DYNE BIO, Gyeonggi-do, Korea) and loaded on an agarose gel. Then, 5 μL of 100 bp marker (DYNE BIO) was also loaded onto the gel. After performing electrophoresis at a voltage of 100 V for 30 minutes, the bands were evaluated using Gel Doc. After the band evaluation, 5 μL of PCR product was mixed with 2 μL of ExoSAP (Thermo Scientific), and the mixture was amplified by PCR at a temperature of 37° C. for 15 minutes and at a temperature of 85° C. for 15 minutes. The insertion of the epitope into VP1 was confirmed by whole DNA sequencing. After confirming the base sequence, the colonies were placed in 200 mL of LB medium containing ampicillin and cultured with shaking at a temperature of 37° C. overnight. A plasmid was prepared using Midi prep (MACHEREYNAGEL, Duren, Germany).

### 2. Preparation of Immune-Boosting Recombinant Foot-and-Mouth Disease Viruses

The recombinant plasmids prepared above containing the recombinant foot-and-mouth disease viruses were transfected into BHKT7-9 (a cell line expressing T7 RNA polymerase) using Lipofectamine 3000 Reagent (Invitrogen, Carlsbad, CA, USA), and then the viruses were cultured for 2 to 3 days and recovered. The prepared viruses were then delivered to fetal goat tongue (ZZ-R) cells or baby hamster kidney-21 (BHK-21) cells for virus propagation.

3. Purification of Antigens from Recombinant Foot-and-Mouth Disease Viruses Type O and A (Inactivated Virus) Presenting C3d-Epitope on Surface

Purified antigens were prepared in BHK-21 cells infected with recombinant immunostimulatory FMDV O PA2-C3d and A22-C3d, the FMDVs being made through recombination with a gene corresponding to the rapidly emerging P1 phenotype (referenced sequence) by reverse genetics according to the modified method described by Lee et al. (Non-patent Document 2).

For virus infection, the culture medium was replaced with Dulbecco's Modified Eagle's medium (DMEM; HyClone, Logan, UT, USA) which was serum-free, and the cells were cultured at a temperature of 37° C. for 1 hour under a 5% CO₂ atmosphere and inoculated with the viruses. Extracellular viruses were then removed. 24 hours after infection, the viruses were inactivated by treatment with 0.003 N binary ethyleneimine twice for 24 hours in a shaking incubator and then concentrated with polyethylene glycol (PEG) 6000 (Sigma-Aldrich, St. Louis, MO, USA). The virus concentrates were layered on sucrose with a 15% to 45% density gradient and centrifuged. After the ultracentrifugation, a hole was made in the bottom of the centrifuge tube, and 1 mL of fractions were collected. The presence of FMDV particles in samples of each fraction was confirmed by optical density using a lateral flow device (BioSign FMDV Ag; Princeton BioMeditech, Princeton, NJ, USA). Before the use of the cells in field experiments, the ZZ-R and BHK-21 cells were treated with the pre-PEG treated supernatant more than twice to confirm that cytopathic effect (CPE) did not occur, so it was confirmed that there was no live virus in the supernatant.

### 4. Identification of Structural and Non-Structural Proteins Using Purified Antigens and Inspection of 146S Particles Using TEM

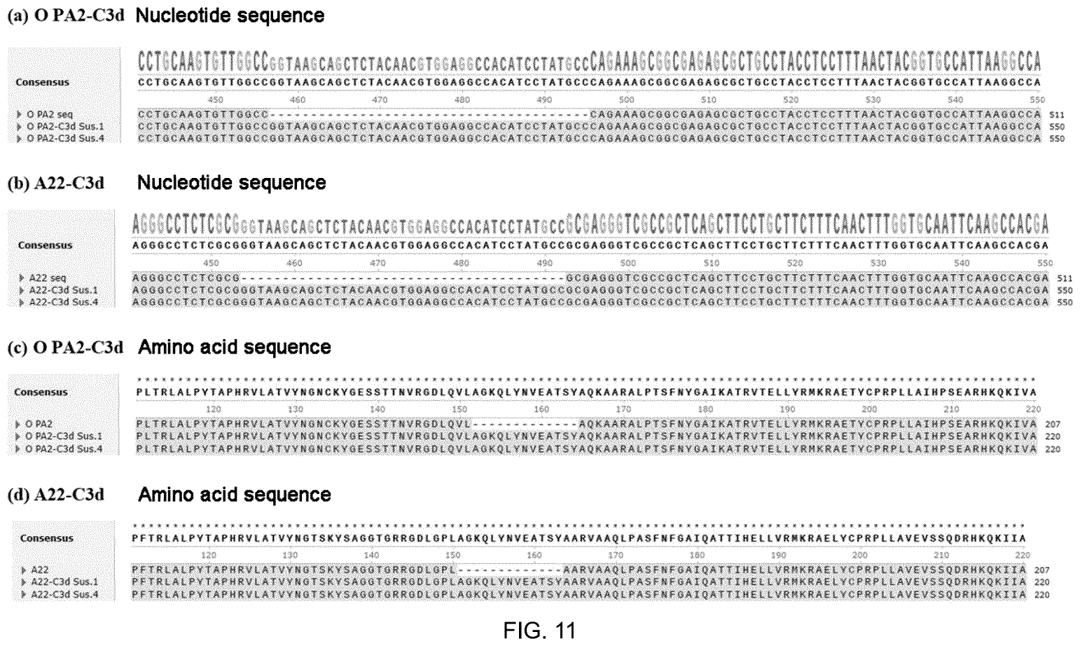

Structural proteins (SPs) expressed using purified antigens from cells infected with immune-boosting recombinant FMDV O PA2-C3d and A22-C3d could be recognized by the formation of bands representing the SPs, and the bands were identified with a Rapid antigen kit (PBM kit, PBM Co Ltd., Princeton, NJ, USA). There was no band formation corresponding to the non-structural proteins (NSPs) of the FMDVs. The characteristics of the virus particles (146S) were confirmed by transmission electron microscopy (TEM) imaging. Additionally, when delivering the recombinant viruses, the sequence often changed. To prove that the sequence did not change during the first and fourth delivery, the FMDVs with the specific epitope of ‘C3d’ inserted were examined to see if the FMDVs maintained the genetic stability of the viruses even after the delivery thereof into cells, and it was confirmed that there was no change in sequence until the fourth passage (FIG. 11).

### 5. Immunogenicity Evaluation of O PA2-C3d and A22-C3d, Immune-Boosting Foot-and-Mouth Disease Vaccine Strains for Overcoming Interference of Maternally-Derived Antibodies in Test Animal (Mice)

### (1) Mice

Mouse experiments were performed according to the method described by Lee et al. (Non-patent Document 2). Age-matched and gender-matched wild-type C57BL/6 mice (6-week-old to 7-week-old female) were purchased from KOSA BIO Inc. (Gyeonggi, Korea). All mice were housed in micro isolator cages with ad libitum access to food and water in an animal facility compliant with the Specific Pathogen-Free (SPF) Animal Biosafety Level 3 (ABSL3) of the Animal and Plant Quarantine Agency. All animals were allowed to acclimate for at least 1 week before being used in experiments. A breeding room was set up with a 12-hour light/dark cycle, a temperature of approximately 22° C., and a relative humidity of approximately 50%. The test was conducted in accordance with institutional guidelines and approval from the Institutional Animal Care and Use Committee under the Animal and Plant Quarantine Agency (certification number IACUC-2021-584).

### (2) Vaccination and FMDV Administration to Mice

To verify the immunogenicity and short-term immunity-inducing effect of antigens isolated and purified from highly immunogenic FMDV O PA2-C3d and A22-C3d and confirm potentials thereof as master seed viruses (MSVs) for developing a foot-and-mouth disease vaccine, animal experiments were conducted as follows.

The vaccine compositions used in the experiments were as follows: O PA2-C3d and A22-C3d (15 μg/dose/mL, 1/10 to 1/640 dose for pigs), ISA 206 (Seppic, Paris, France, 50%, w/w), 10% Al(OH)₃ and 15 μg/mouse Quil-A (InvivoGen, San Diego, CA, USA). FMDV (100 LD 50 ME-SA topotype from O/VET/2013 or 100 LD₅₀ A/Malay/97, SEA topotype) was injected intramuscularly (IM) into the thigh muscles of mice at 0 days post vaccination (0 dpv) and injected intraperitoneally (IP) to the same mice at 7 dpv. Mice in the negative control group were administered with the same volume of phosphate-buffered saline (PBS, pH 7.0) through the same routes. Survival rates and body weight changes were monitored for up to 7 days post-challenge (dpc) after the administration to assess short-term immunogenicity.

As a preliminary experiment for the experiments in the target animal (pigs), a PD₅₀ test was conducted to confirm the immunogenicity of the bivalent test vaccine including antigens of O PA2-C3d antigen+A22-C3d. The target animal group was compared with a group administered with a test vaccine including antigens of O PA2 antigen+A22, which was used as the backbone of the immune-boosting vaccine strains. The vaccine compositions used in the experiments were as follows; O PA2-C3d antigen+A22-C3d antigens (15 μg+15 μg/dose/ml, 1/10 to 1/640 dose) or O PA2 antigen+A22 antigens (15 μg+15 μg/dose/ml, 1/10 to 1/640 dose), ISA 206 (50%, w/w), 10% Al(OH)₃, 15 μg Quil-A/mouse. The negative control group was administered with the same volume of PBS through the same routes.

After the mice were vaccinated at 0 dpv via an I.M. injection, the mice were intraperitoneally administered with FMDVs (100 LD₅₀, O/VET/2013, ME-SA topotype or 100 LD₅₀ A/Malay/97, SEA topotype) at 7 days post vaccination (7 dpv), and then survival rates and body weight changes were monitored until 7 days post challenge (dpc).

### 6. Immunogenicity Evaluation of O PA2-C3d and A22-C3d, Immune-Boosting Foot-and-Mouth Disease Vaccine Strains for Overcoming Interference of Maternally-Derived Antibodies in Target Animal (Pigs)

### (1) Pigs

Pigs (8 to 9 weeks old, total n=32) were screened using SP O ELISA and SP A ELISA for antibody titers (PI value: 50%) and VN titers (based on 1.6 Log₁₀), and then divided into MDA(+) and MDA(−) groups (n=16 for each group).

The pigs in each group of the MDA(+) and MDA(−) groups were randomly divided into three groups (n=4 or 6/group): a negative control (NC) group, a O PA2+A22 (positive control, PC)-treated group, and a O PA2-C3d+A22-C3d-treated group.

The animals were isolated in a closed isolation room (ABSL3) during the experiments. After arrival at ABSL, all animals were housed in cages with ad libitum access to food and water and used in experiments after at least 1 week of acclimatization. A breeding room was set up with a 12-hour light/dark cycle, a temperature of approximately 22° C., and a relative humidity of approximately 50%. This disclosure was conducted in accordance with institutional guidelines and approval from the Institutional Animal Care and Use Committee under the Animal and Plant Quarantine Agency (certification number IACUC-2021-584).

### (2) Induction of Immune Response and Sampling Through Vaccination

To evaluate the immunogenicity of antigens isolated and purified from O PA2-C3d and A22-C3d, the immune-boosting foot-and-mouth disease vaccine strains manufactured to overcome interference of maternally-derived antibodies in the target animal, which were pigs, and to observe the avoidance effect of overcoming the interference of the maternally-derived antibodies, the experiments were performed using MDA(+) (FMD-seropositive)-wild pigs and MDA(−) (FMD-seronegative)-wild pigs, which were positive and negative for maternally-derived antibodies, respectively, and the vaccine compositions were as follows; A total of 1 ml of vaccine was taken as 1 dose, and the vaccine was prepared by including 15 μg O PA2 antigen+15 μg A22 antigen (positive control group, PC group) or 15 μg O PA2-C3d antigen+15 μg A22-C3d antigen (experimental group), ISA 206 (50%, w/w), 10% Al(OH)₃, and 150 μg Quil-A. The negative control group (NC group) was administered with the same volume of PBS through the same routes.

The pigs at the ages of 8 to 9 weeks, which were positive or negative for maternally-derived antibodies, were screened and divided into two groups: MDA(+) group (n=16) and MDA(−) group (n=16). The individuals in the MDA(+) group and MDA(−) group were divided into three groups: an NC group (PBS-administered group, n=4/group), a PC group (O PA2+A22-administered group, n=6/group), and an experimental group (O PA2-C3d+A22-C3d administration group, n=6/group). Each group was administered with 1 mL vaccine through an I.M. route twice at 28-day intervals (0 dpv and 28 dpv). Blood samples from vaccinated pigs were collected at 0, 7, 14, 28, 42, 56, 70, and 84 dpv and subjected to serological analysis for such as SP O ELISA and SP A ELISA, and VN titer confirmation. In the case of SP O ELISA and SP A ELISA, considering the antibody positivity rate depending on the characteristics of the antigens, FMDV types O and A were compared using the PrioCHECK™ kit and VDPro™ kit, respectively. In addition, peripheral blood mononuclear cells (PBMCs) were isolated from the blood samples collected at all sampling schedules, and changes in gene expression related to test vaccine-mediated cellular and humoral immune responses were analyzed.

### (3) Serological Analysis

To detect SP antibodies in serum, PrioCHECK™ FMDV type O or FMDV type A (Prionics AG, Switzerland) and VDPro® FMDV type O or FMDV type A (Median Diagnostics, Gangwon-do, Korea) were used. The absorbance of the ELISA plates was converted to percent inhibition (PI) values. Animals were considered positive for antibodies when the PI value was greater than or equal to 50% for the PrioCHECK™ FMDV kit or greater than or equal to 40% for the VDPro® FMDV kit.

A virus neutralization test (VNT) was performed in accordance with the manual of the World Organization for Animal Health (OIE). The serum was heat-inactivated at a temperature of 56° C. for 30 minutes in a water bath. Cell density was adjusted to form a 70% monolayer, and two-fold serial dilutions (1:8-1:1024) of serum samples were prepared. The diluted serum samples were then incubated with homologous viruses of 100-tissue culture infectious dose (TCID)₅₀/0.5 mL at a temperature of 37° C. for 1 hour. After 1 hour, LF-BK (bovine kidney) cell suspension was added to all wells. After 2 to 3 days, CPE was assessed to determine a titer, which was calculated as the inverse of antibody dilution in the form of a Log₁₀ value required to neutralize the 100 TCID₅₀ viruses. FMDV O/PA2 and FMDV A22/IRAQ were used for the VNT.

### (4) PECs Isolation and Cell Culture

Naïve mice were anesthetized using CO₂ and sacrificed. The abdominal cavity was washed with 5 mL of balanced salt solution (HBSS, Gibco, Waltham, MA, USA) buffer of Hank, which was cooled without Ca²⁺, Mg²⁺, and phenol-red. A peritoneal lavage fluid was centrifuged at 300×g at a temperature of 4° C. for 10 minutes. The pelletized PECs were resuspended and counted using a Bio-Rad TC20 automated cell counter (Bio-Rad). All cells were freshly isolated before use. Cryopreserved cells were not used in any of the experiments. Then, the purified PECs were incubated in a complete medium. The medium was made from 10% fetal calf serum (HyClone), 3 mM L-glutamine (Sigma-Aldrich), 10 mM HEPES (Sigma-Aldrich), 100 U/mL of penicillin/streptomycin (Sigma-Aldrich), and Roswell Park Memorial Institute (RPMI) 1640 (Gibco, Carlsbad, CA, USA) supplemented with 0.05 mM 2-beta-mercaptoethanol (Sigma-Aldrich). The incubation was performed at a temperature of 37° C. and under a 5% CO₂ atmosphere.

### (5) PBMCs Isolation and Cell Culture

Porcine PBMCs were isolated from the whole blood of pigs vaccinated at the previously mentioned specific time points (n=4 or 6/group) in accordance with the method described by Lee et al. (Non-patent Document 2). The whole blood (20 mL/individual) was collected independently in BD Vacutainer heparin tubes (BD, Becton, Dickinson and Company, Franklin Lakes, NJ, USA). The PBMCs were isolated using Ficoll-Paque™ PLUS (GE Healthcare Bio-Sciences Corp., Piscataway, NJ, USA) gradient centrifugation. Residual red blood cells were lysed by treatment with ammonium-potassium chloride (ACK) lysis buffer (Gibco, Carlsbad, CA, USA). The PBMCs were suspended in Dulbecco's PBS (Gibco) without Ca²⁺ and Mg²⁺. The PBMCs were then supplemented with 2% fetal bovine serum (FBS) (Gibco) and counted using a volumetric flow cytometer (Miltenyi Biotec, Bergisch Gladbach, Germany). All cells were freshly isolated before use. Cryopreserved cells were not used in any of the experiments. Afterward, the purified PBMCs were resuspended in RPMI1640 (Gibco) medium supplemented with 10% FBS (HyClone, Logan, UT, USA), 3 mM L-glutamine (Sigma-Aldrich, St. Louis, MO, USA), and 100 U/mL of penicillin-streptomycin (Sigma-Aldrich).

The PBMCs were suspended in DPBS (Gibco) without Ca²⁺ and Mg²⁺. The PBMCs were then counted using a Bio-Rad TC20 automated cell counter (Bio-Rad). All cells were freshly isolated before use. Cryopreserved cells were not used in any of the experiments. Afterward, the purified PBMCs were resuspended in RPMI-1640 (Gibco) medium supplemented with 10% FBS (Gibco), 3 mM L-glutamine (Sigma-Aldrich), 10 mM HEPES (Sigma-Aldrich), and 100 U/mL of penicillin-streptomycin (Sigma-Aldrich). The incubation was performed at a temperature of 37° C. and under a 5% CO₂ atmosphere.

### (6) Antigen-Induced IFNγ ELISpot Assay for PECs and PBMCs In Vitro

PA2-C3d antigen-mediated and A22-C3d antigen-mediated IFNγ secretions were analyzed using a commercial ELISpot assay kit (Catalog numbers EL485 and EL985 for mice and pigs, R&D Systems, Minneapolis, MN, USA) in accordance with the manufacturer's instructions. Isolated murine PECs or porcine PBMCs (5×10⁵ cells/well) were cultured in 96-well PVDF-supported microplates containing monoclonal capture antibodies specific for mouse or porcine IFNγ. Inactivated FMDV (O PA2, O PA2-C3d, A22, and A22-C3d) antigens were stimulated at each concentration of 4 μg/mL (final concentration) for 18 hours in a humidified incubator at a temperature of 37° C. and under a 5% CO₂ atmosphere. PBS and 5 μg/mL of phorbol myristate acetate (PMA, Sigma-Aldrich) were used as negative and positive controls, respectively. The plates were washed with wash buffer and incubated with biotinylated anti-mouse IFNγ antibodies (1:119) or anti-porcine antibodies (1:119) at a temperature of 4° C. overnight. Afterward, the plates were incubated with streptavidin (1:119) AP-conjugated at RT for 2 hours. The plates were washed and developed with 5-bromo-4-chloro-3′indoliphosphate p-toluidine salt (BCIP)/nitro blue tetrazolium chloride (NBT). The cells were counted using an ImmunoSpot ELISpot reader (AID iSpot reader system; Autoimmune Diagnostika GmbH, Strassberg, Germany). Results were expressed as spot forming units (SFU).

(7) RNA Isolation, cDNA Synthesis, and Quantitative Real-Time PCR

Total RNAs were extracted from purified porcine PBMCs using a TRIzol reagent (Invitrogen) and RNeasy Mini Kits (QIAGEN, Valencia, CA, USA). cDNAs were prepared by reverse transcription using a GoScript reverse transcription system (Promega, Madison, WI, USA) in accordance with the manufacturer's instructions. The synthesized cDNAs were amplified by quantitative real-time PCR (qRT-PCR) on a Bio-Rad iCycler using iQ SYBR Green Supermix (BioRad, Hercules, CA, USA).

Gene expression levels were normalized to hprt levels and presented as relative ratios compared to controls. The list of primers used in this disclosure is listed in Table 1.

TABLE 1
List of primer sequences for qRT-PCR

| | | | Length | Sequence |
|---|---|---|---|---|
| Target | Forward/Reverse | Sequence (5′-3′) | (mer) | number |
| IFNα | IFNα F | CATCTGCTCTCTGGGCTGTG | 20 | 14 |
| | IFNα R | TGAGGGGATCCAAAGTCCCT | 20 | 15 |
| IFNß | IFNß F | TGCAACCACCACAATTCCAGA | 21 | 16 |
| | IFNß R | GGTTTCATTCCAGCCAGTGC | 20 | 17 |
| IFNγ | IFNγ F | GCCATTCAAAGGAGCATGGAT | 21 | 18 |
| | IFNγ R | CTGATGGCTTTGCGCTGGAT | 20 | 19 |
| IL-1β | IL-Iβ F | AGCCAGTCTTCATTGTTCAGGT | 22 | 20 |
| | IL-Iβ R | TCATCTCTTTGGGGCCATCAG | 21 | 21 |
| IL-17A | IL-17A F | CTCGTGAAGGCGGGAATCAT | 20 | 22 |
| | IL-17A R | GGTGTGCTCCGGTTCAAGAT | 20 | 23 |
| IL-23p19 | IL-23p19 F | CCATATCCAGTGCGGGGATG | 20 | 24 |
| | IL-23p19 R | AGGCCTTGGTGGATCCTTTG | 20 | 25 |
| IL-23R | IL-23R F | TCCCTCATTGCAAAGCACAA | 20 | 26 |
| | IL-23R R | GCATCTCCTCTTGCAAGCAAAT | 22 | 27 |
| IL-2 | IL-2 F | AAGCTCTGGAGGGAGTGCTA | 20 | 28 |
| | IL-2 R | CAACAGCAGTTACTGTCTCATCA | 23 | 29 |
| IL-10 | IL-10 F | CGGCCCAGTGAAGAGTTTCT | 20 | 30 |
| | IL-10 R | TGCCTTCGGCATTACGTCTT | 20 | 31 |
| TGFβ | TGFβ F | GGCTGTCCTTTGATGTCACC | 20 | 32 |
| | TGFβ R | GGCCAGAATTGAACCCGT | 18 | 33 |
| IL-4 | IL-4 F | CTCACCTCCCAACTGATCCC | 20 | 34 |
| | IL-4 R | TGTGTCCGTGGACGAAGTTG | 20 | 35 |
| IL-6 | IL-6 F | CTGCAGTCACAGAACGAGTG | 20 | 36 |
| | IL-6 R | CGGCATCAATCTCAGGTGCC | 20 | 37 |
| CD40 | CD40 F | GTCATCAGCACAAATACTGC | 20 | 38 |
| | CD40 R | CACAAGTGGTGTCTGTTTTC | 20 | 39 |
| CD80 | CD80 F | TCAGGCATCGTTCAGGTGAC | 20 | 40 |
| | CD80 R | TGACAGCCAGCACCATTTCA | 20 | 41 |
| CD86 | CD86 F | TGGGACTGAGTAACATTCTCTTTGT | 25 | 42 |
| | CD86 R | CCAGCTCATCCAGGCTTAGG | 20 | 43 |
| MHC Class | MHC Class I F | TGAGCTATTTCTACACCGCCG | 21 | 44 |
| I | MHC Class I R | TCGTCCACGTAGCCGACTT | 19 | 45 |
| MHC Class | MHC Class II F | CTCCAGTGATGCTGGGTCAG | 20 | 46 |
| II | MHC Class II R | TGACAGAGTGCCCGTTCTTC | 20 | 47 |
| CD21 | CD21 F | TGCCATGCCTACAAAGCTGA | 20 | 48 |
| | CD21 R | GTAGTAACCAGGGCGGCATT | 20 | 49 |
| CD28 | CD28 F | TCAAAGGAGTTCCGGGCATC | 20 | 50 |
| | CD28 R | CTGAAGCAGGCGGGAGTAAT | 20 | 51 |
| ICOS | ICOS F | GGATGTGCAGCCTTTGTTGT | 20 | 52 |
| | ICOS R | CAGAGCGTACCAAATTGCGG | 20 | 53 |
| CTLA4 | CTLA4 F | GAGTATGGGTCTGCAGGCAA | 20 | 54 |
| | CTLA4 R | ATATGTCGCGGCACAGACTT | 20 | 55 |
| AHNAK | AHNAK F | CACCATCACCGTGACTCGAA | 20 | 56 |
| | AHNAK R | AGTTCGTGCCGTGGAATCTT | 20 | 57 |
| HPRT | HPRT F | CCCAGCGTCGTGATTAGTGA | 20 | 58 |
| | HPRT R | GCCGTTCAGTCCTGTCCATA | 20 | 59 |

### 7. Statistics

All quantitative data were expressed as mean±standard error of the mean (SEM) unless otherwise specified. Statistical significance between groups was assessed using two-way ANOVA followed by Tukey's post hoc test or one-way ANOVA followed by Tukey's post hoc test. *p<0.05; **p<0.01; ***p<0.001; and ****p<0.0001. Parametric tests were used to compare different groups. Survival curves we re created using the Kaplan-Meier method, and differences were analyzed using the log-rank sum test. GraphPad Prism 9.1.2 (GraphPad, San Diego, CA, USA) software and IBM SPSS software (IBM Corp., Armonk, NY, USA) were used for all statistical analyses.

### <Example> Manufacturing of Immune-Boosting Foot-and-Mouth Disease Vaccine Strains for Overcoming Interference of Maternally-Derived Antibody and Preparation and Purification of Inactivated Antigens Using O PA2-C3d and A22-C3d

To develop a foot-and-mouth disease vaccine for overcoming the interference of maternally-derived antibodies, the PI backbones of the O1 Manisa-O PA2 (O1 M-O PA2) and O1 Manisa-A22/Iraq/24/64 (O1 M-A22) strains, which were previously developed using reverse genetics, were used. In the case of type O, based on the results of previous studies that confirmed the vaccine matching rate for surrounding outbreak situations and the proliferation of the viruses in floating cells, especially based on the results of previous studies that confirmed antigen-mediated immunogenicity in the test animal (mice) and target animal (pigs), O PA2 was determined to be the strongest candidate vaccine strain. In the case of type A, when looking at the global outbreak situation, the matching rate generally tends to be low, but A22 was also classified as a suitable vaccine strain.

In the present disclosure, a strategy to prepare foot-and-mouth disease vaccine strains for overcoming the interference of maternally-derived antibodies against FMDV type O and FMDV type A by inserting the active site of C3d, which was a B cell epitope into the O PA2 and A22 P1 backbone was the same as shown in FIGS. 1a and 1b, and detailed methods are described in the Materials and methods part.

### <Experiment Example 1> Evaluation of Immunogenicity in Mice of FMD Vaccine Including Antigens of O PA2-C3d and A22-C3d, Immune-Boosting Foot-and-Mouth Disease Vaccine Strains for Overcoming Interference of Maternally-Derived Antibodies

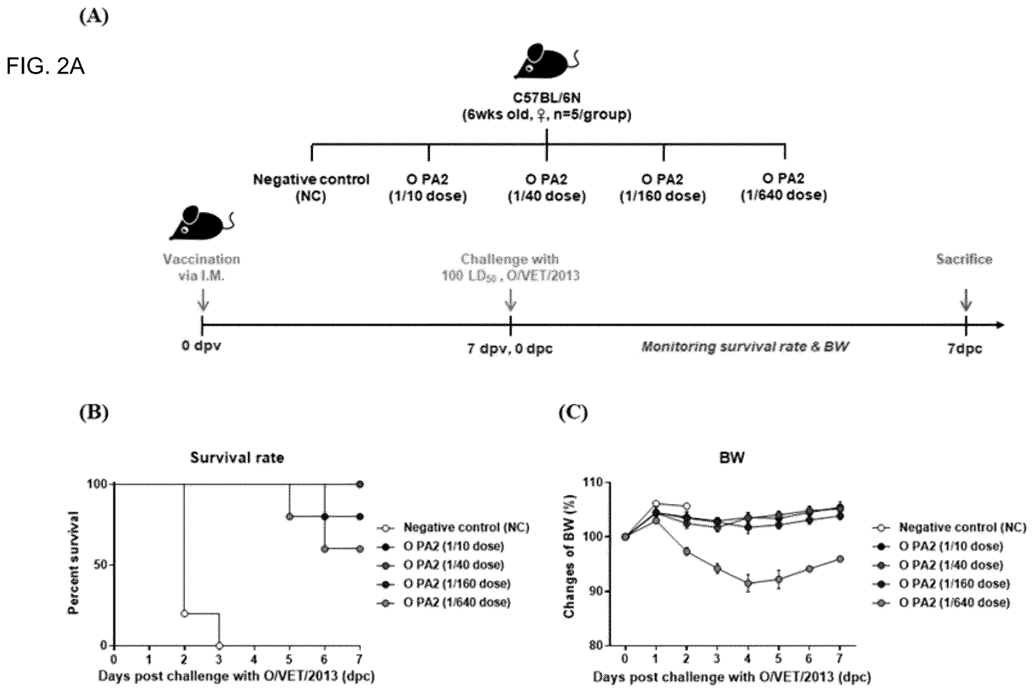

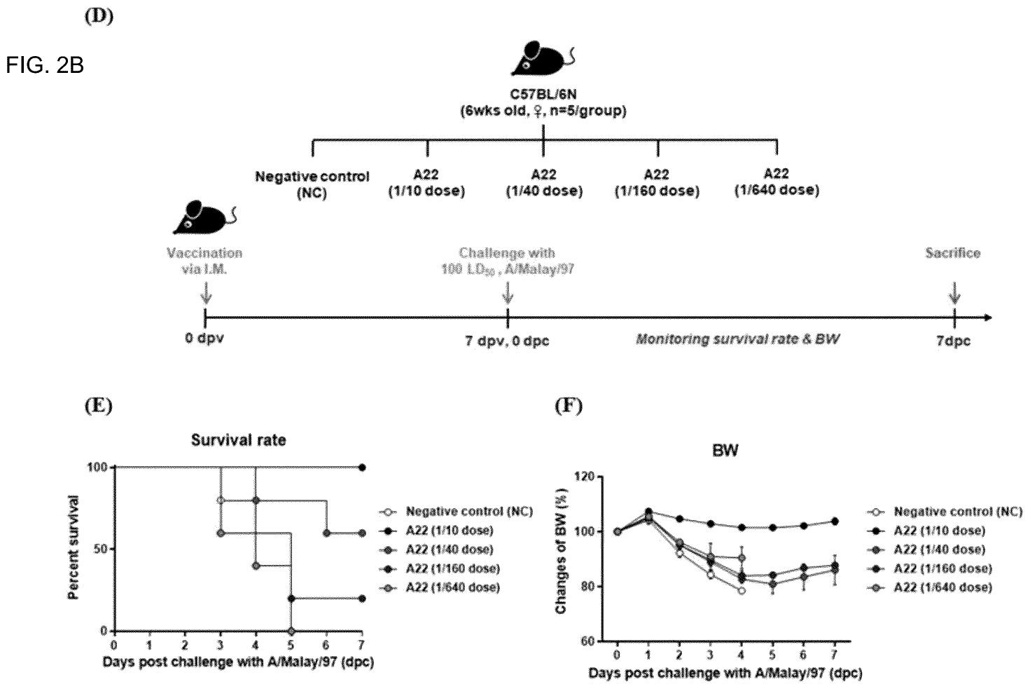

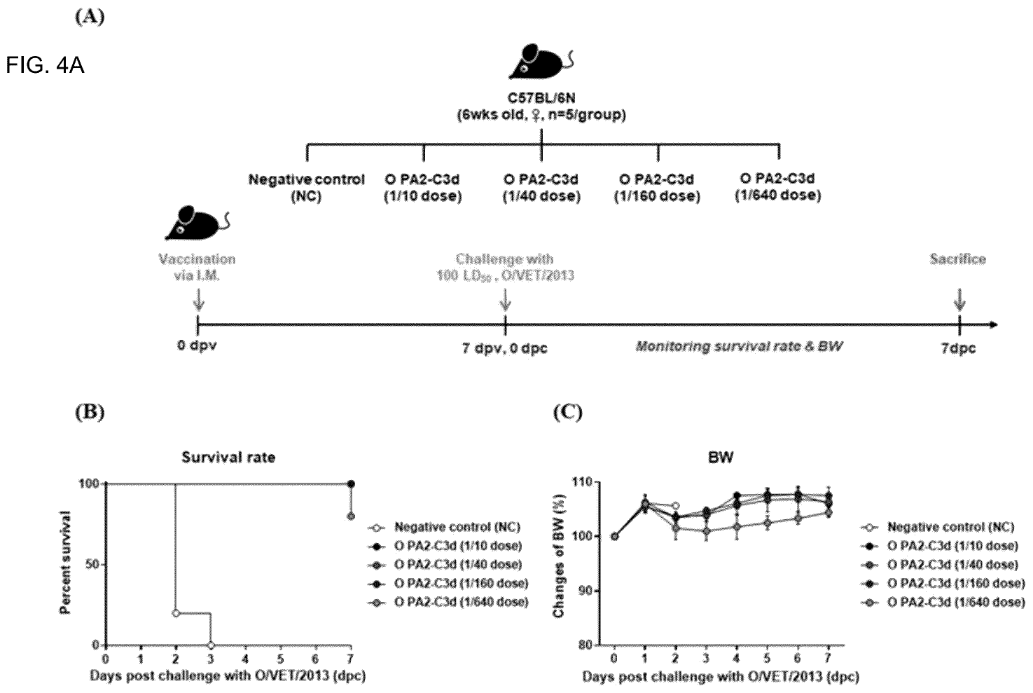

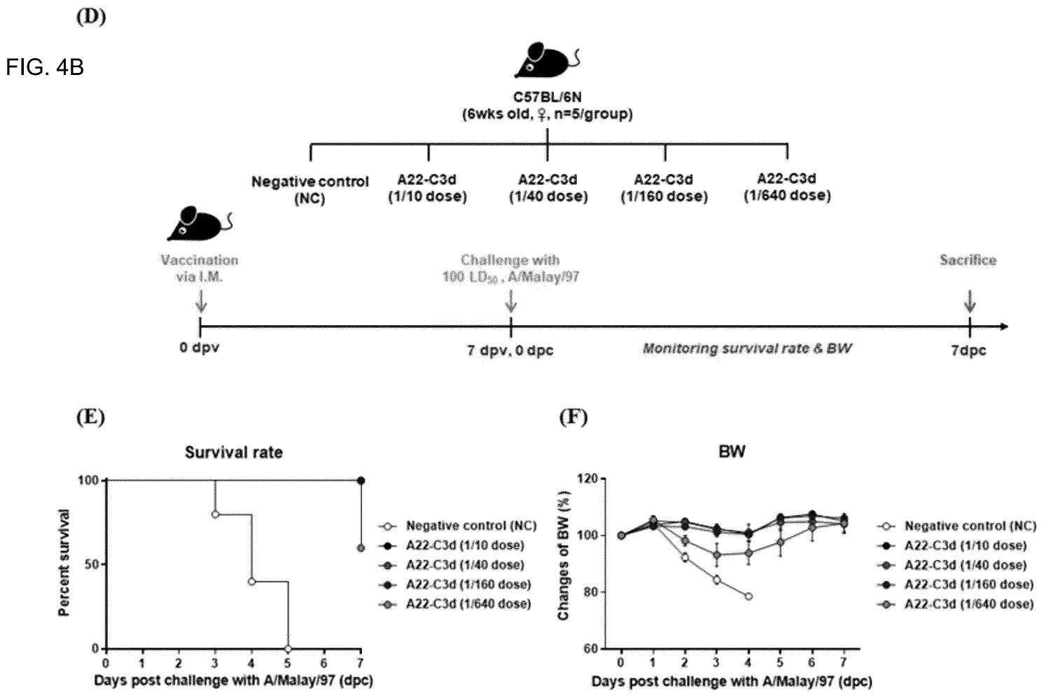

To confirm the immunogenicity of antigens isolated and purified from O PA2-C3d and A22-C3d, which had B cell epitope C3d inserted for activation of B cells, to confirm the immunogenicity of antigens isolated and purified from O PA2 and A22 in mice, to evaluate the potential as a master seed virus (MSV) of vaccines for foot-and-mouth disease, and to evaluate the protective effect against FMDV infection, experiments involving O PA2-C3d and A22-C3d were performed with the same strategy as shown in (A) in FIG. 4a and (D) in FIG. 4b, respectively, and experiments involving O PA2 and A22 were performed with the same strategy as shown in (A) in FIG. 2a and (D) in FIG. 2b. The vaccine compositions used in the experiments were as follows; O PA2-C3d or A22-C3d antigens, O PA2 or A22 antigens (15 μg/dose/mL, 1/10 to 1/640 dose), ISA 206 (50%, w/w), 10% Al(OH)₃, 15 μg Quil-A/mouse.

After the mice were vaccinated at 0 days post vaccination (0 dpv) via an intramuscular (I.M.) injection, the mice were intraperitoneally injected with FMDVs (100 LD 50, O/VET/2013, ME-SA topotype or 100 LD₅₀ A/Malay/97, SEA topotype) at 7 dpv, and then survival rates ((B) and (C) in FIG. 4a) and body weight changes ((E) and (F) in FIG. 4b) were monitored until 7 days post challenge (dpc).

As a result of the experiments, the test vaccine containing the antigens of O PA2-C3d showed 97.01 PD₅₀ (Log₄) with survival rates of 100% at 1/10, 1/40, and 1/160 doses and 80% at 1/640 dose in mice, and little weight loss was observed at the 1/10, 1/40, and 1/160 doses ((B) and (C) in FIG. 4a). In addition, when the test vaccine using the antigens isolated and purified from A22-C3d was inoculated into mice, the test vaccine showed 73.52 PD₅₀ (Log₄) with survival rates of 100% at 1/10, 1/40, and 1/160 doses and 60% at 1/640 dose. In terms of weight loss, almost no change in body weight was observed at 1/10, 1/40, and 1/160 doses ((E) and (F) in FIG. 4b).

However, the vaccine including the antigens of O PA2 showed 55.72 PD₅₀ (Log₄) ((B) of FIG. 2a). The body weight change of O PA2 was reduced to a lower level compared to O PA2-C3d ((C) in FIG. 2a).

The vaccine including the antigens of A22-C3d showed 73.52 PD₅₀ (Log₄) with survival rates of 100% at 1/10, 1/40, and 1/160 doses and 60% at 1/640 doses. No change in body weight was observed at 1/10, 1/40, and 1/160 doses.

However, the vaccine including the antigens of A22 showed 6.06 PD₅₀ (Log₄) ((E) of FIG. 2b). The body weight change of A22 was reduced to a lower level compared to A22-C3d ((F) in FIG. 2b).

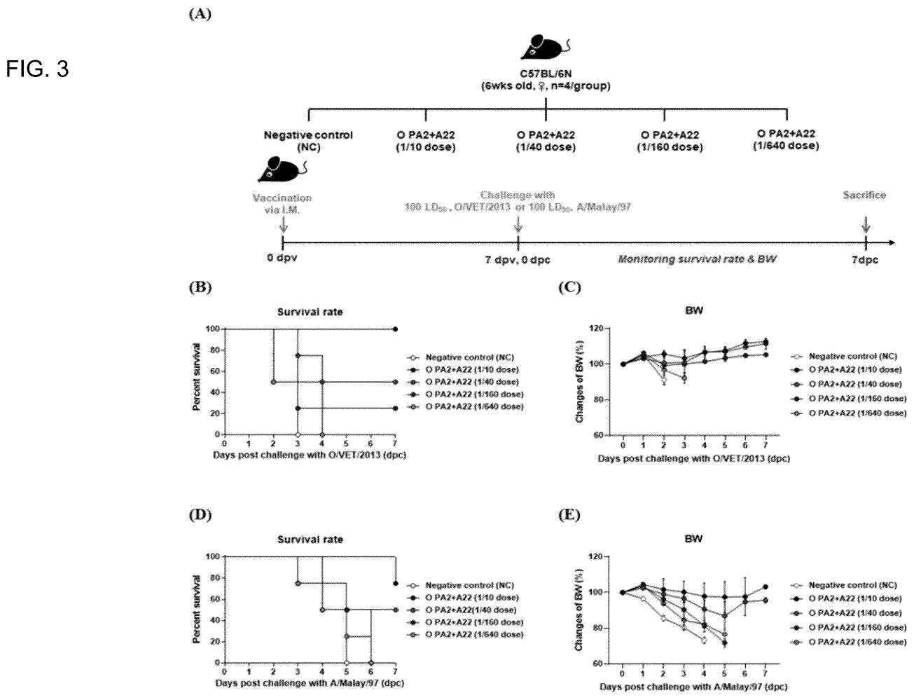

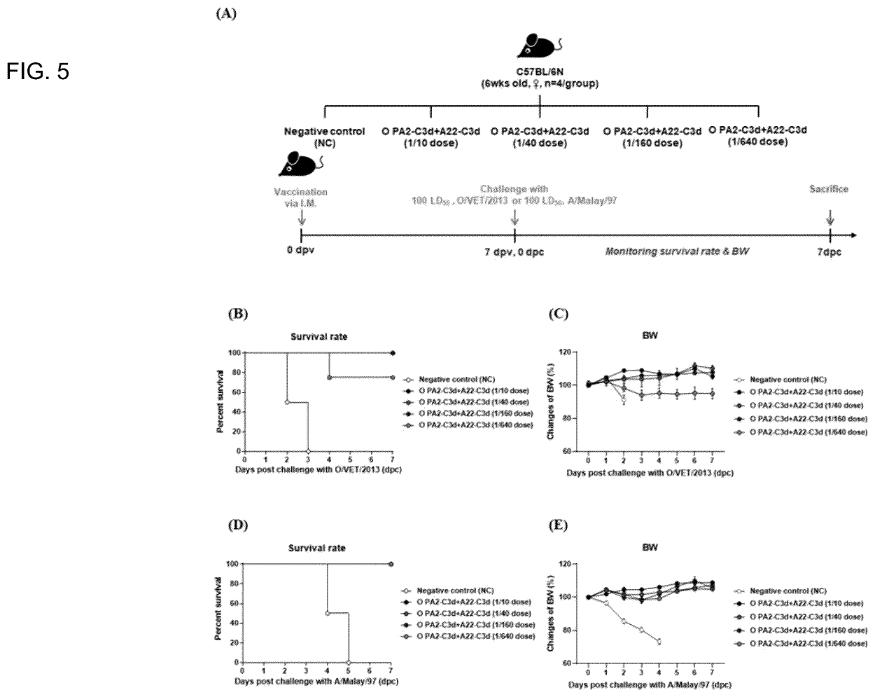

A PD₅₀ test was performed to determine the immunogenicity of the investigational bivalent vaccine (including O PA2-C3d+A22-C3d antigens and via a combined administration of O PA2-C3d and A22-C3d) in pigs. Results were compared with those of the group that received the test vaccine (including the antigens of PA2+A22 and via a combined administration of O PA2 and A22) used as the backbone of the immune-boosting vaccine strain (FIGS. 5 and 3).

With the same experimental strategies as shown (A) in FIG. 5 and (A) in FIG. 3, mice were vaccinated via an I.M. injection at 0 dpv, and the mice were challenged with FMDVs (100 LD 50 from O/VET/2013, ME-SA topotype or 100 LD 50 from A/Malay/97, SEA topotype) administered via an I.P. Injection thereto at 7 dpv. Survival rates and body weight changes were monitored from 0 days post challenge (0 dpc) to 7 dpc.

The bivalent vaccine including the antigens of O PA2+A22 showed PD₅₀ (Log₄) values of 5.66 and 4 when the mice were challenged with O/VET/2013 and A/Malay/97, respectively ((B) and (D) in FIG. 3).

The bivalent vaccine including the antigens of O PA2-C3d+A22-C3d showed PD₅₀ (Log₄) values of 90.5 and >128 when the mice were challenged with O/VET/2013 and A/Malay/97, respectively ((B) and (D) in FIG. 5).

### <Experiment Example 2> Evaluation of Immune Effect (Early, Mid-Term, Long-Term) in Target Animal (Pigs) of FMD Vaccine Including Antigens of PA2-C3d and A22-C3d, Immune-Boosting Foot-and-Mouth Disease Vaccine Strains for Overcoming Interference of Maternally-Derived Antibodies

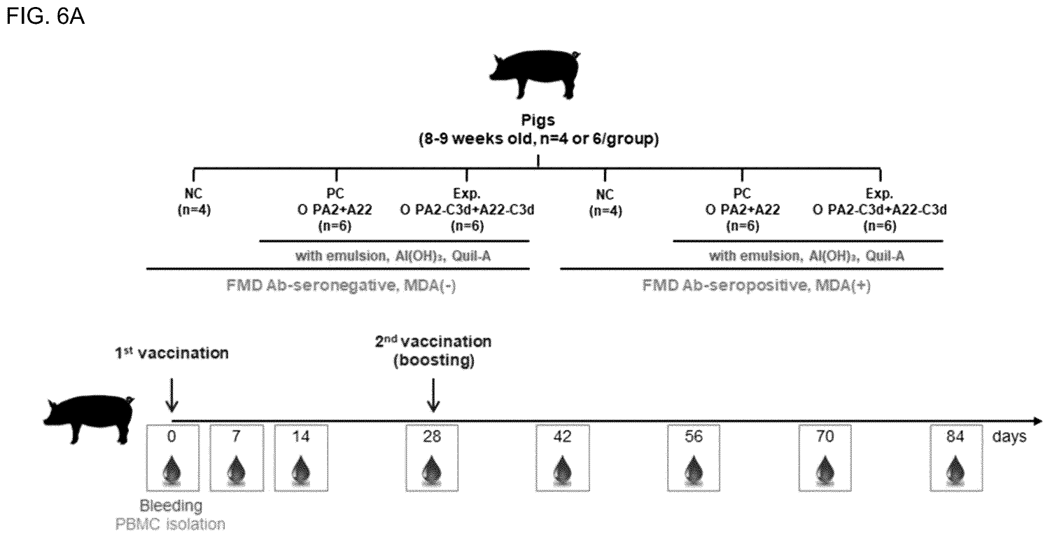

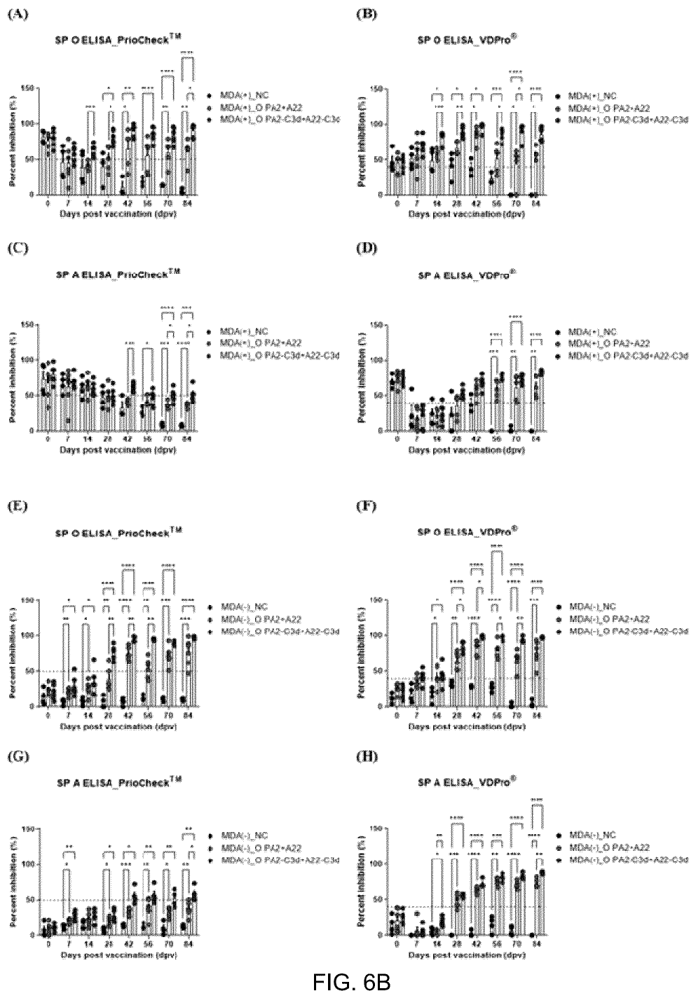

To evaluate the immunogenicity of antigens isolated and purified from O PA2-C3d and A22-C3d, the immune-boosting foot-and-mouth disease vaccine strains manufactured to overcome the interference of maternally-derived antibodies in the target animal, which was pigs and to observe the avoidance effect of overcoming the interference of maternally-derived antibodies, the experiments were performed using MDA(+) (FMD-seropositive)-wild pigs and MDA(−) (FMD-seronegative)-wild pigs, which were positive and negative for maternally-derived antibodies, respectively (FIG. 6a).

As a result, when administering a test vaccine using the antigens of O PA2-C3d+A22-C3d to pigs in the MDA(+) group to confirm the effect of overcoming the interference of maternally-derived antibodies, from 14 dpv, the antibody titer by SP O ELISA in the MDA(+) group significantly increased compared to the PC group vaccinated with the test vaccine including the antigens of O PA2+A22 (p<0.001, PrioCheck™ kit, VDPro® kit), and even at 28 dpv, the antibody titer of the MDA(+) group showed significance of p<0.05 (PrioCheck™ kit) and p<0.01 (VDPro® kit) ((A) and (B) in FIG. 6b). In particular, after the second vaccination (boosting) of the test vaccine at 28 dpv, the antibody titer was very high in the experimental group. Meanwhile, in the NC group, maternally-derived antibodies continued to decrease. At 56, 70, and 84 dpv, the antibody titers of the experimental group and the NC group had a difference of p<0.0001 or p<0.001. Additionally, in the case of SP A ELISA in the MDA(+) group, at 42 dpv, the antibody titer of the experimental group was higher than the antibody titer of the PC group (p<0.001, PrioCheck™ kit). At 56, 70, and 84 dpv, significant differences between the experimental group and the NC group were observed at the p<0.5, p<0.0001, p<0.001 (PrioCheck™ kit), and p<0.0001 (VDPro® kit) levels, respectively ((C) and (D) in FIG. 6b).

Meanwhile, when administering the test vaccine to the MDA(−) group to confirm the immunogenicity and induction of early, mid-term, and long-term immunity in the target animal (pigs) of the test vaccine including the antigens of O PA2-C3d+A22-C3d, the antibody titer by SP O ELISA in the experimental group significantly increased compared to the NC group at the p<0.05 level at 7 dpv (PrioCheck™ kit) and 14 dpv (VDPro® kit), and from 28 dpv to 84 dpv, the antibody titer of the experimental group showed significance compared to the NC group (p<0.0001) ((E) and (F) in FIG. 6b). Looking at the antibody titers by SP A ELISA for each group in the MDA(−) group, the results were the same as the results shown by SP O ELISA. There was a significant difference in the antibody titers between the experimental group and the NC group at 7 dpv (PrioCheck™ kit) and 14 dpv (VDPro® kit) (p<0.01 and p<0.05), and from 28 dpv to 84 dpv, the experimental group showed a higher antibody titer than the NC group (p<0.0001, p<0.001, p<0.01, and p<0.05). In addition, the antibody titer of the experimental group was consistently higher than that of the PC group. The antibody titers of difference of the two groups had a significant difference observed at p<0.01 and p<0.05 (VDPro® kit) at 14 and 84 dpv ((G) and (H) in FIG. 6b).

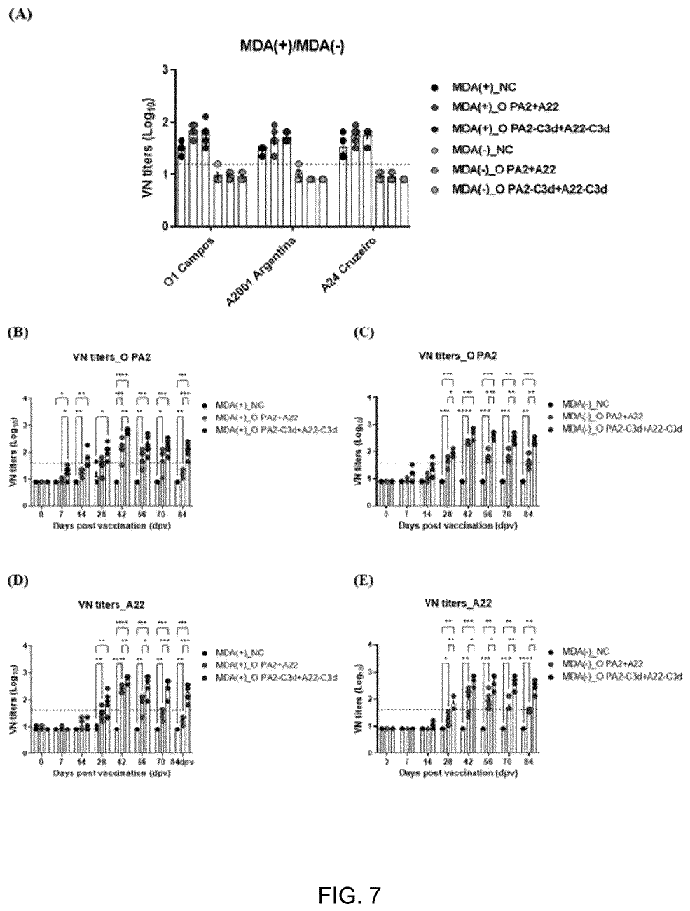

Before vaccination (0 dpv), neutralizing antibody titers against O1 Campos, A2001 Argentina, and A24 Cruzeiro in the MDA(+)/MDA(−) groups are shown (A) in FIG. 7. In the MDA(+) group, the neutralizing antibody titer was measured to be >1.6 Log₁₀, and in the MDA(−) group, the neutralizing antibody titer was measured to be <1.2 Log₁₀. The neutralizing antibody titers were confirmed using homologous viruses for O PA2 and A22, which were the backbone viruses used as antigens in the PC group ((B) and (C) in FIG. 7). In the case of the MDA(+) group, the antibody titers by SP O ELISA and SP A ELISA at 0 dpv were high, but the neutralizing antibody titers against O PA2 and A22 were low below the protective level ((B) in FIG. 7). This is believed to be due to the fact that the mother pigs of the piglets with high maternally-derived antibodies (MDA(+)) received the vaccine from Company B, showing low antibody titers against heterologous viruses such as O PA2 and A22.

In the case of the MDA(−) group, the neutralizing antibody titer against O PA2 ((C) in FIG. 7) was significantly higher in the experimental group, which was administered with the test vaccine including the antigens of O PA2-C3d+A22-C3d from 7 dpv, than in the NC and PC groups (p<0.05). Afterward, the neutralizing antibody titer of the experimental group continued to increase until 14 and 28 dpv (p<0.01 and p<0.05), and after boosting at 28 dpv, the neutralizing antibody titer increased higher compared to the NC group from 42 dpv to 84 dpv (p<0.0001, p<0.001, p<0.01, and p<0.05). In addition, even after boosting, the neutralizing antibody titer of the experimental group was higher than that of the PC group, and the antibody titers between the two groups had a significant difference observed at 42 dpv and 84 dpv (p<0.001 and p<0.01).

Looking at the neutralizing antibody titers against A22 in the MDA(+) and MDA(−) groups ((D) and (E) in FIG. 7), the neutralizing antibody titers in the experimental group at 28 dpv were higher than in the NC and PC groups (p<0.01), and after boosting, from 42 dpv to 84 dpv, the neutralizing antibody titers of the experimental group increased to a higher level than those of the NC group (p<0.0001 and p<0.001). The neutralizing antibody titers of the experimental group and the PC group had a significant difference from 42 dpv to 84 dpv (p<0.0001 and p<0.001).

### <Experiment Example 3> Evaluation of Induction of Cellular Immune Responses in Pigs by FMD Vaccine Including Antigens of O PA2-C3d and A22-C3d, Immune-Boosting Foot-and-Mouth Disease Vaccine Strains for Overcoming Interference of Maternally-Derived Antibodies

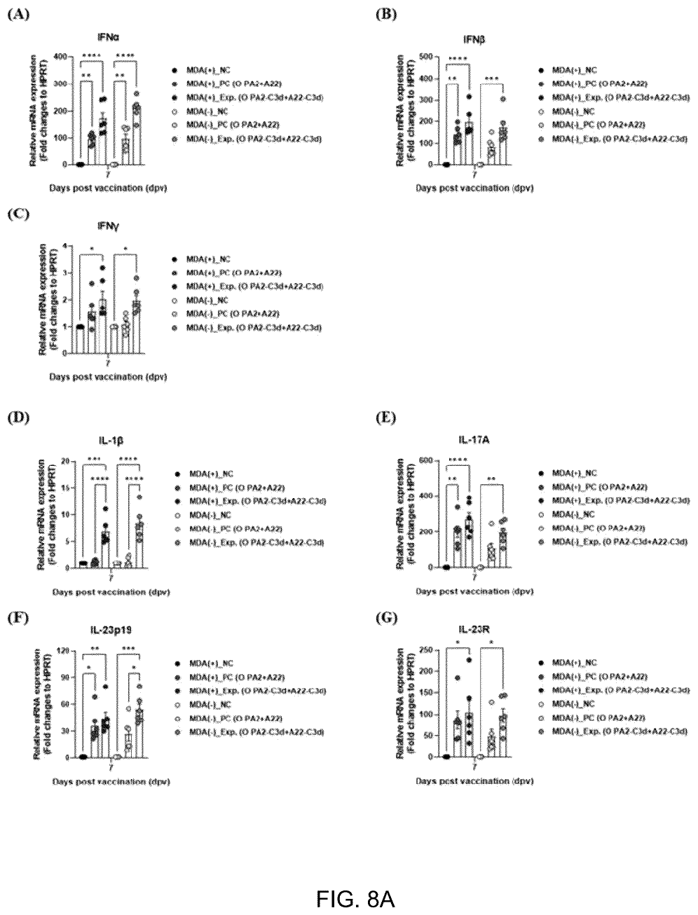

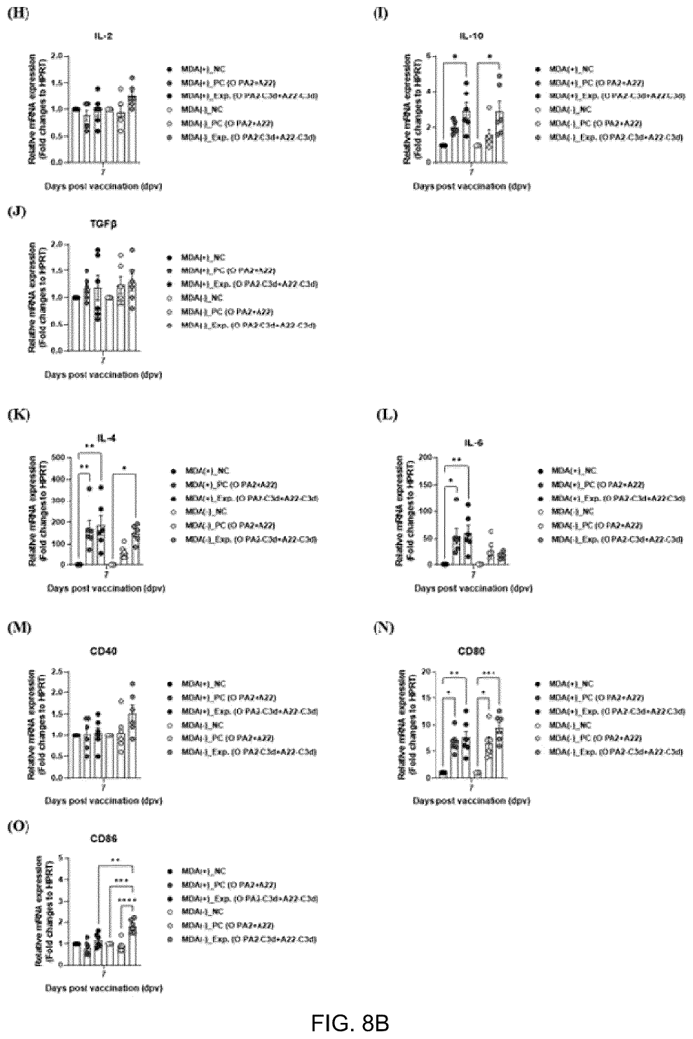

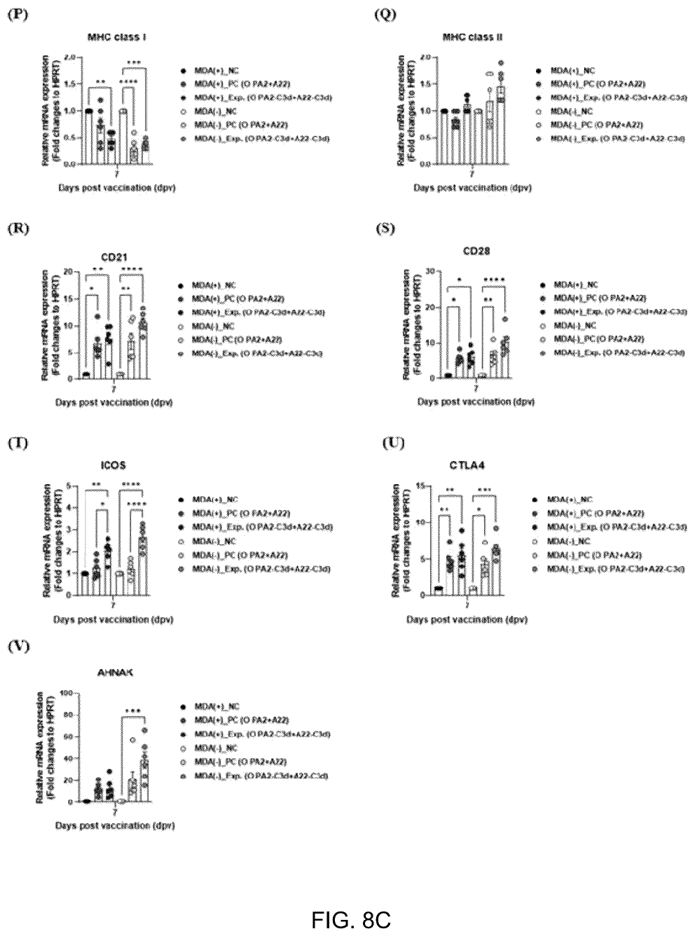

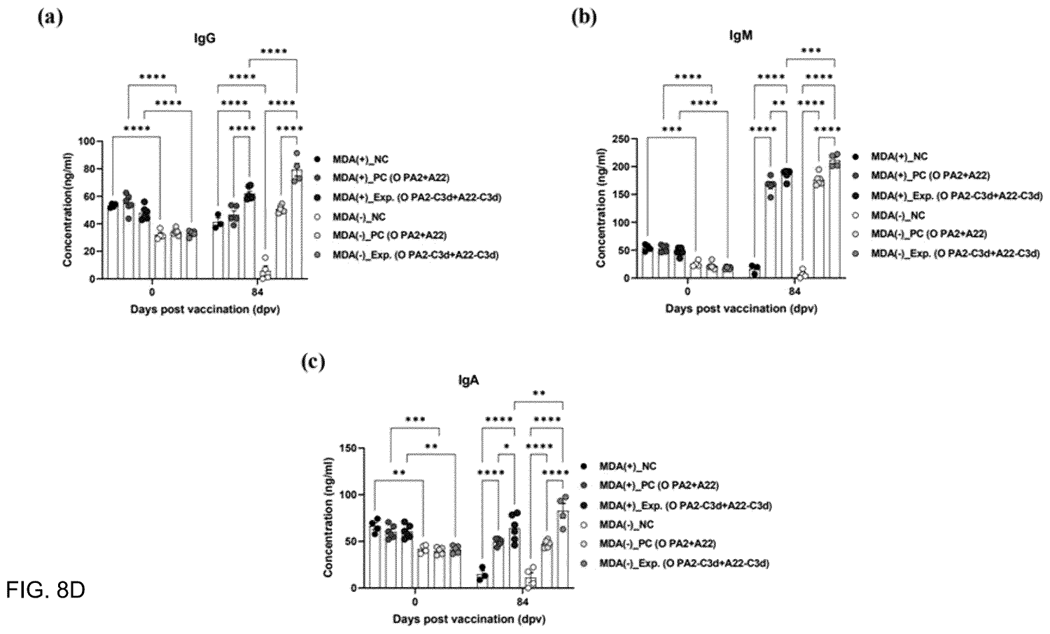

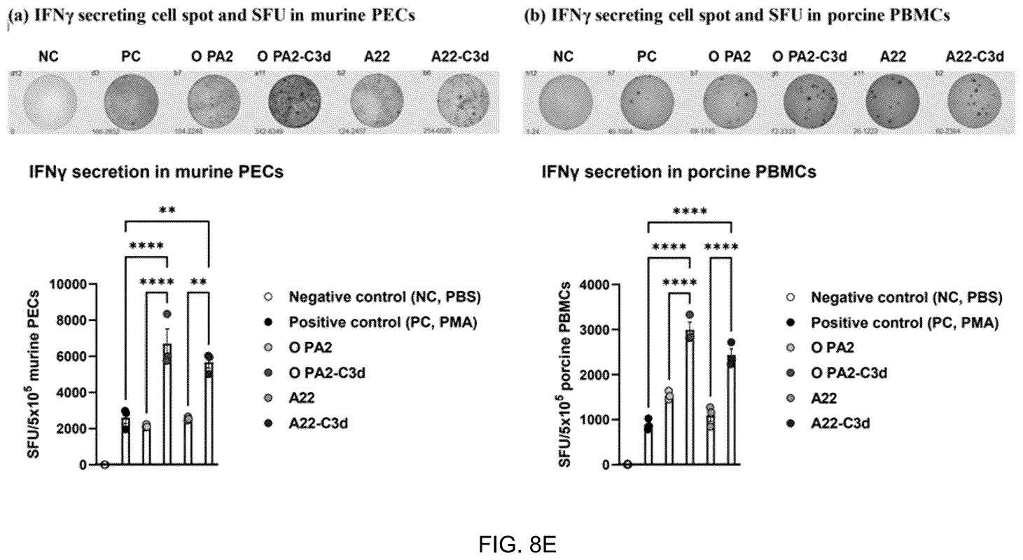

3-1. As shown in FIG. 6a, after vaccinating the target animal, which was pigs, with a test vaccine including the antigens of O PA2-C3d and A22-C3d, at each sampling time, PBMCs were isolated from the whole blood of the pigs, and qRT-PCR was performed. Afterward, observed were changes in the expression of genes for cytokines (IFNα, IFNβ, IFNγ, IL-1β, IL-17A, IL-23p19, IL-23R, IL-2, IL-10, TGFβ, IL-4, and IL-6) and co-stimulatory molecules (CD40, CD80, CD86, MHC class I, MHC class II, CD21, CD28, CTLA4, ICOS, and AHNAK), both of which were related to the induction of cellular immune responses (FIGS. 8a to 8c).

As a result, overall, the expression of proinflammatory cytokine genes was observed to be very high. In particular, when experimenting with individuals in the MDA(+)/MDA(−) conditions, the expressions of type I IFNs, IFNα, and IFNβ had a very high increase of the p<0.0001 and p<0.001 levels in the experimental group compared to the NC group at 7 dpv. Meanwhile, looking at the difference between the PC group and the NC group, the expression of IFN was higher in the PC group than in the NC group in both MDA(+)/MDA(−) conditions (p<0.01). However, the expression of IFNβ in the PC group had a significant increase compared to the NC group only in the MDA(+) condition (p<0.01). The expression of IFNγ was somewhat lower than the expressions of IFNα and IFNβ, but a significantly higher expression was observed in the experimental group compared to the NC group in both MDA(+)/MDA(−) conditions (p<0.05).

The expression of IL-1β was significantly higher in the experimental group than in the NC group in MDA(+)/MDA(−) conditions (p<0.0001 and p<0.001), in particular, compared to the PC group, the expression of IL-1B in the experimental group showed a significant difference at the p<0.0001 level. The expression of IL-17A was much higher in the experimental group than in the NC group in MDA(+)/MDA(−) conditions (p<0.0001 and p<0.01), and the significance in the expression of IL-17A was observed between the PC and NC groups in the MDA(+) condition (p<0.01). The expression of IL-23p19 was also significantly higher in the experimental group than in the NC group in MDA(+)/MDA(−) conditions (p<0.01 and p<0.001), in particular, the expression of IL-23p19 between the experimental group and PC group was significantly different in the MDA(−) condition (p<0.05). The expression of IL-23R was also very high, as were the expressions of other cytokines. In both MDA(+)/MDA(−) conditions, the expression of IL-23R showed a significant difference between the experimental group and the NC group (p<0.05). The expression levels of IL-4 and IL-6 were higher in the MDA(+) condition than in the MDA(−) condition. In the MDA(+) condition, the significance in the expression of IL-4 and IL-6 was at p<0.01 between the experimental group and the NC group. The significance in the expression of IL-4 and IL-6 was at p<0.01 and p<0.01, respectively, between the PC group and the NC group. In the MDA(−) condition, the expression level of IL-4 between the experimental group and the NC group had a significant difference observed at p<0.05. Meanwhile, the expression of IL-10, which was an anti-inflammatory cytokine, was found to be higher in the experimental group than in the NC group in MDA(+)/MDA(−) conditions (p<0.05). The expression levels of IL-2 and TGFβ were higher in the order of the experimental group>the PC group>the NC group, but no significance was observed between each group.

The expression levels of co-stimulatory molecules were overall somewhat lower than the expression levels of cytokines, but it was confirmed that the expression levels of co-stimulatory molecules were significantly increased in the experimental group compared to the PC group or NC group. The expression of CD80, CD21, CD28, CTLA4, and ICOS tended to increase upon vaccine administration in the MDA(−) condition compared to the MDA(+) condition. The expression of CD80, CD21, CD28, CTLA4, and ICOS showed significance between the experimental group and the NC group in MDA(+)/MDA(−) conditions (p<0.0001, p<0.001, p<0.01, and p<0.05). The expressions of the remaining genes except ICOS between the PC group and the NC group showed significance observed at the p<0.01 and p<0.05 levels. Among the gene expressions, the expressions of CD80, CD21, and CD28 showed very high changes due to test vaccine administration. The expression of ICOS between the experimental group and the PC group showed significance at p<0.01 in the MDA(+) condition and p<0.0001 in the MDA(−) condition, which means the expression of ICOS was higher in the C3d-inserted strain compared to the backbone strain.

The expressions of CD86 and AHNAK between the experimental group and NC group in the MDA(−) condition showed significance (p<0.0001 and p<0.001), in particular, the expression of CD86 between the experimental group and the PC group showed a significant difference (p<0.0001). Meanwhile, the expression level of MHC class I was found to be reduced in the MDA(−) condition compared to the MDA(+) condition. The expression of CD40 and MHC class II between each group showed no significance.

3-2. Humoral immune responses mediated by the immune-boosting foot-and-mouth disease virus types O and A (O PA2-C3d and A22-C3d) according to the present disclosure, as measured by immunoglobulin subtypes such as IgG, IgM, and IgA in pigs are as follows.

Pigs (8 to 9 weeks old) that were FMD antibody-negative (MDA(+), n=16) or FMD antibody-negative (MDA(−), n=16) were divided into three groups each. A negative control group (NC, n=4/group), a positive control group (PC, n=6/group), and an experimental group (Exp., n=6/group).

The experimental group was administered with a test vaccine including 15 μg (1 dose for cattle and pigs) of O PA2-C3d+A22-C3d antigen plus ISA 206 (oil-based emulsion, 50%, w/w), 10% Al(OH)₃, and 150 μg Quil-A. The positive control (PC) group was administered with a test vaccine including 15 μg (1 dose for cattle and pigs) of O PA2-C3d+A22-C3d antigen plus ISA 206 (oil-based emulsion, 50%, w/w), 10% Al(OH)₃, and 150 μg Quil-A. The negative control (NC) group was administered with the same volume of PBS as a test vaccine. Vaccination was performed twice at a 28-day interval. 1 mL vaccine (1 dose) was injected into the test animal's neck via a deep intramuscular route. For serological assays, blood samples were collected from the pigs at 0, 7, 14, 28, 42, 56, 70, and 84 days after vaccination, and the experimental results are shown in FIG. 8d.

In FIG. 8d, (A) shows IgG concentration; (B) shows IgM concentration; and (C) Shows IgA concentration. Data represent mean±SEM of triplicate measurements (n=4 or 6/group). Statistical analysis was performed using two-way ANOVA followed by Tukey's test. *p<0.05; **p<0.01; ***p<0.001; and ****p<0.0001.

As a result of the experiments, O PA2-C3d and A22-C3d were effectively induced in MDA(+)/MDA(−) animals when with not only SP-specific antibody titers (via SP ELISA) but also SP-non-specific antibodies including IgG (indicator of neutralizing antibodies), IgM (first natural antibody induced during pathogen infection or vaccination), and IgA (core element inducing mucosal immunity) levels. Maternal IgG and IgA may attenuate mucosal helper cell responses in early infancy, and maternal IgG regulatory T cell epitopes induce immune tolerance rather than immunogenicity. Based on the results, the recombinant immune-boosting foot-and-mouth disease vaccine strains O PA2-C3d and A22-C3d can effectively induce active immunity in the host by overcoming MDA interference-mediated immune tolerance in MDA(+) animals. In addition, the vaccine strains may induce robust cellular and humoral immune responses when vaccinated into MDA(−) animals or animals at the ages of 2 to 3 months (period of lower MDA titers depending on the current vaccination program). These results suggest that C3d spikes on antigens directly activate B cells by persistently stimulating B cell surface receptors and that the highly immunogenic O PA2-C3d and A22-C3d antigens provide a robust T cell-mediated immune response, generating more efficient high titers and neutralizing antibodies (FIG. 8d).

From the results, it was determined that O PA2-C3d and A22-C3d, as immune-boosting foot-and-mouth disease vaccine strains, have excellent immunogenicity of the antigens themselves and play an important role in inducing short-term immunity and initial host defense. In addition, it is expected that the O Bivalent test vaccine including the antigens of PA2-C3d antigen+A22-C3d will be more effective in overcoming the interference of maternally-derived antibodies by simultaneously inducing a robust cellular immune response and humoral immune response in the early stage when vaccinating the target animal.

### <Experiment Example 4> Evaluation of Effectiveness in Pigs of Overcoming Interference of Maternally-Derived Antibodies and Simultaneously Inducing Cellular and Humoral Immune Responses

To evaluate the effectiveness of overcoming the interference of maternally-derived antibodies and simultaneously inducing cellular and humoral immune responses in pigs, experiments involving MDA(+)- or MDA(−)-pigs were performed (FIG. 6a). In the MDA(+)-individuals, O PA2-C3d+A22-C3d especially showed a strong effect of overcoming maternally-derived antibodies through antibody titer induction by SP O ELISA. In the case of antibody titer by SP A ELISA, the antibody titer itself decreased somewhat at the beginning of vaccination and then tended to increase after boosting. This is determined that due to the nature of the FMDV type A antigens, the FMDV type A antigens have a different SP region from the antigen coated on the SP A ELISA plate, so the detection itself appeared low, affecting the percent inhibition (PI) value. For a more accurate evaluation, it was deemed necessary to review whether it was possible to overcome the interference of maternally-derived antibodies through VN titers.

Meanwhile, in the MDA(−)-individuals, O PA2-C3d+A22-C3d induced a strong increase in antibody titers by SP O ELISA and SP A ELISA, and the ability to induce antibody titers was significant compared to O PA2+A22 used as backbone (FIG. 8b).

Additionally, to confirm the actual virus neutralization effect, VN titers for O PA2 and A22 were confirmed. Since the individuals in question were vaccinated with a vaccine from Company B (the source of the commercialized vaccine is not listed to protect the company's rights and interests and prevent disputes), to confirm the presence of maternally-derived antibodies, VN titers for O1 Campos, A2001 Argentina, and A24 Cruzeiro were confirmed using a serum at 0 dpv before vaccination. As a result, the VN titers for the MDA(+) group and MDA(−) group were accurately classified as positive and negative, respectively, and vaccination was performed for each group using these animals.

As a result of confirming the VN titers for O PA2 and A22 using the serum from 0 to 84 dpv at each blood collection time, the antigens of the vaccine strains in the vaccines inoculated into mother pigs were different from the antigens in the vaccines inoculated in the present disclosure, and thus regardless of the MDA(+)/MDA(−) groups, VN titers for O PA2 and A22 were <1.2 Log₁₀, which is below the defense level in the initial stage (0 dpv). In both maternally-derived antibody positive/negative groups, the administration of the O PA2-C3d+A22-C3d bivalent test vaccine showed significantly high VN titers compared to the administration of the O PA2+A22 bivalent test vaccine (FIG. 7).

From the results, O PA2-C3d and A22-C3d, the immune-boosting foot-and-mouth disease vaccine strains developed to overcome the interference of maternal-derived antibodies, are expected to effectively induce active immunity in the host by overcoming interference phenomena such as immune tolerance even in the presence of maternally-derived antibodies. In addition, depending on the antivirus program currently in use, the vaccine strains are believed to induce a robust humoral immune response even when inoculated into the target animal (pigs) at the ages of 8 to 12 weeks, when maternally-derived antibodies are decreasing. This is determined that since C3d spiked on the antigen continuously stimulates B cell surface receptors to directly activate B cells, while the highly immunogenic O PA2-C3d antigens and A22-C3d antigens induce a strong T cell-mediated cellular immune response, more efficient production of high titer antibodies and neutralizing antibody titers is possible.

To prove this, a bivalent test vaccine including the antigens of O PA2-C3d+A22-C3d was used to confirm the O PA2-C3d+A22-C3d antigen-mediated cellular immune response in MDA(+)/MDA(−) pigs. The positive control (PC) group received a bivalent test vaccine including the antigens of the backbone strain O PA2+A22, and the negative control (NC) group received PBS. In both MDA(+)/MDA(−) conditions, the test vaccine including the antigens of O PA2-C3d+A22-C3d induced a very high level of expression of type I IFN, which had an antiviral effect at the beginning of vaccination (7 dpv). Thus, the test vaccine was expected to be able to effectively protect the host in the early stages of vaccination against FMDV infection. In the case of IFNγ, a T helper (Th) 1 cell-related cytokine, the expression level thereof was lower than the expression levels of IFNα and IFNβ, but a significant expression of >2 fold-change (p<0.05) was shown, so it was determined that IFNγ effectively induced T cell-mediated cellular immune responses. It was found that the expression of IL-1B, which is involved in the activity of inflammasomes, the expression of Th17 cells, and the expression of IL-17A derived from unconventional T cells (γδ T cells) were also very high in the experimental group inoculated with the antigens isolated and purified from the C3d-inserted vaccine strains. Through previous research by the current inventors, the expressions of IL-23p19 and IL-23R were found to be very important in the host's initial defense. In such a situation, in the present disclosure, the IL-23p19 and IL-23R were initially expressed at the level of a ‘cytokine storm’ and then the expressions appeared to normalize.

When IL-23A is secreted through stimulation of pathogen recognition receptors (PRRs) in innate immune cells such as dendritic cells (DCs) and macrophages (M^{Φ}S), IL-23A binds to IL-23R on the surface of non-traditional T cells, which are innate-like immune cells, and stimulates the cells to produce IL-17A. The produced IL-17A plays a critical role in the host's initial defense by recruiting neutrophils to the site of pathogen infection, forming a neutrophil extracellular trap (NET), and causing NETosis of the pathogen.

In addition, the IL-23/IL-17A axis is known to link innate immunity and adaptive immunity. It is believed that the test vaccine including the antigens of O PA2-C3d+A22-C3d simultaneously induces innate and adaptive immune responses through the secretion of these pro-inflammatory cytokines.

TGFβ, which is known to play a central role in the differentiation and survival of CD4⁺ Th subsets and CD4⁺ T regulatory cells (T_{regs}) and to be involved in the development of T_{regs} and the induction of immunological tolerance in DCs with IL-2, a T cell growth factor essential for the generation of memory cells, had a slight high expression in the experimental group, but no significance was observed between each group. The expression of IL-10, an anti-inflammatory cytokine, was also significantly increased in the experimental group (p<0.05), which is presumed to be due to host homeostasis to control the ‘cytokine storm’ of inflammatory cytokines. The expressions of Th2 cell-derived cytokines, IL-4 and IL-6, were higher in the MDA(+) group than in the MDA(−) group, and the expressions of these cytokines increased in the presence of passive immunity by maternally-derived antibodies.

It is determined that CD80 and CD86 co-stimulatory signaling, which cooperates with T cell receptor (TCR) signaling to promote activation of T cells, increased in the O PA2-C3d+A22-C3d administration group, thereby the immune-boosting foot-and-mouth disease vaccine strains can effectively stimulate T cells by effectively presenting antigens to T cells.

In the case of MHC class I, gene expression was higher in the MDA(+) group than in the MDA(−) group and lower in the vaccinated group than in the NC group. It is determined that in the presence of passive immunity, MHC class I gene expression appeared to be low, thereby inhibiting antigen recognition by cytotoxic CD8⁺ T cells in the early stages of vaccination. Meanwhile, MHC class II had a higher expression thereof in the MDA(−) group compared to the MDA(+) group, and although no significance between groups was shown, the expression of MHC class II tended to increase in the experimental group. From this, it is determined that the antigens of the C3d inserted immune-boosting vaccine strains activate CD4⁺ T cells, which induce cooperation and regulation of effector cells through the presentation of MHC class II by APC (DCs, M^{Φ}s, and B cells). It is determined that the antigens of the C3d-inserted immune-boosting vaccine strains may induce the formation of continuous cell-cell contact and activation of T cells through interaction with the MHC complex.

Meanwhile, the expression of CD21, a direct C3d receptor, was significantly increased in both MDA(+)/(MDA(−) conditions by vaccination with a test vaccine containing the antigens of C3d-inserted foot-and-mouth disease vaccines (p<0.01 and p<0.0001), thereby activation of B cells appeared to be possible through stimulation of C3d on the surface of FMDVs and binding to CD21.

The expressions of CD28 and ICOS, co-stimulatory signals that are co-stimulated upon T cell activation and play an important role in the induction of memory T cells, increased significantly in MDA(+)/MDA(−) conditions by vaccination with the test vaccine containing the antigens of O PA2-C3d+A22-C3d (CD28: p<0.0001; p<0.05; ICOS: p<0.01; p<0.00001). In particular, the expression of ICOS induced a significant difference between the experimental group and the PC group (MDA(+): p<0.05; MDA(−): p<0.0001). It appears that the antigens of C3d-inserted foot-and-mouth disease vaccine strains increased co-stimulation of T cells and lymphocytes and induced significant expression of IFNγ. Additionally, ICOS is known to affect the intestinal immune relationship for IgA production as an immunoglobulin domain, thereby ICOS is expected to be used for a foot-and-mouth disease vaccine in the future to simultaneously induce systemic immunity and mucosal immunity. The expression of CTLA4 also showed a similar trend to the expression of ICOS. The CTLA pathway is thought to be induced by the expression of ICOS, meanwhile, when administering foot-and-mouth disease vaccines to overcome the interference of maternally-derived antibodies in the cytoplasmic domain, the induced CTLA4 causes the conversion of regulatory T cells to suppress autoimmunity by expressing pro-inflammatory cytokines at the level of a ‘cytokine storm’.

Meanwhile, in the present disclosure, the expression of AHNAK was significantly increased in the O PA2-C3d+A22-C3d administration group in the MDA(−) condition (p<0.001) and was somewhat higher in the experimental group in the MDA(+) condition. However, no significance was observed between each group. AHNAK is a large 700 kDa protein previously identified as a structural scaffold protein. AHNAK has been involved in various cellular processes such as cell structure, intracellular trafficking, cell membrane regeneration, regulated exocytosis, T cell differentiation, and calcium signaling pathways during T cell activation.

Cytolytic CD8⁺ T cells (CTLs) kill virus-infected cells in a calcium-dependent manner. AHNAK is expressed in mature CTLs, but not in naïve CD8⁺ T cells. It has been reported that calcium introduction is very important for proper function to induce an immune response. In fact, ANHAK-deficient (Ahnak1^{-/-}) CTL caused a significant decrease in Granzyme B production, cytolytic activity, and IFNγ secretion after TCR stimulation.

Thus, it is thought that the test vaccine including the antigens of O PA2-C3d+A22-C3d can induce T cell activation and CTL response through the expression of AHNAK.

In addition, to verify that the antigens of immune-boosting foot-and-mouth disease viruses according to the present disclosure induce secretion of IFNγ, an indicator of cellular immune response and to verify a specific cellular immune response to be induced by the C3d-inserted FMDV antigens, Ag-mediated secretion of IFNγ by ‘C3d’ inserted FMDVs was confirmed through in vitro ELISpot analysis using peritoneal exudate cells (PECs) isolated from mouse peritoneal lavage fluid and peripheral blood mononuclear cells (PBMCs) isolated from pig whole blood.

As a result, the antigens of the inactivated FMDVs from O PA2-C3d and A22-C3d induced significantly higher levels of IFNγ secretion than control groups on mouse PECs and porcine PBMCs (FIG. 8e). All in all, these results demonstrate that O PA2-C3d and A22-C3d may induce a Th1-type immune response.

### <Experiment Example 5> Evaluation of Possibility of Measuring Antigen Content in a Simple Kit and Differentiating Vaccine Strains from Wild Strains

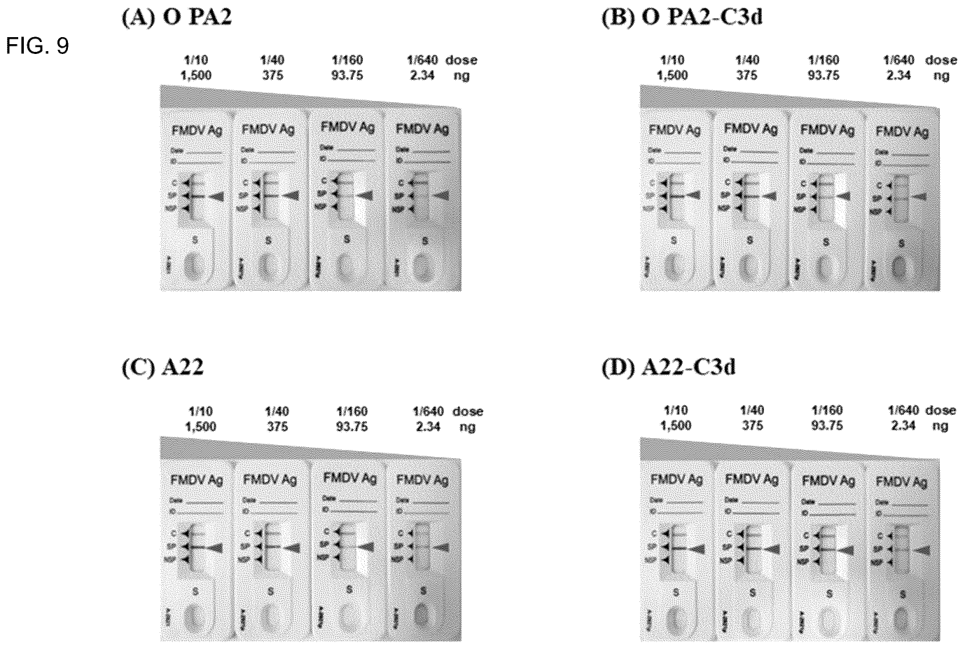

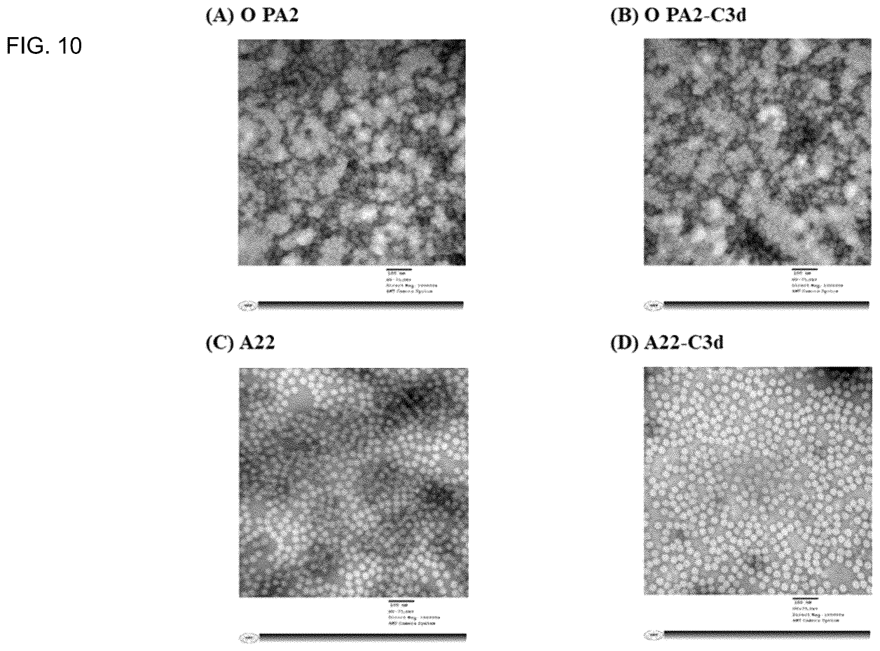

Inactivated antigens were produced and purified using two types of recombinant foot-and-mouth disease viruses, O PA2-C3d and A22-C3d, prepared according to the present disclosure and using two types of backbone viruses, O PA2 and A22, for positive control (PC) group. As a result of measuring the antigen content using a simple kit and performing a differentiation test from wild strains using these virus strains, a positive SP band was confirmed even with a small amount of 2.34 ng (1/640 dose), and it was found that it was possible to differentiate the virus strains from wild strains through the absence of an NSP band (FIG. 9). As a result of electron microscopy (TEM) observation of the antigens (146s particles) purified through a sucrose density gradient, it was confirmed that there was no problem in the formation of 146s particles, thereby the antigens were used as antigens for a foot-and-mouth disease test vaccine (FIG. 10).

1. A recombinant plasmid comprising a sequence represented by SEQ ID NO: 8.

2. A recombinant immune-boosting foot-and-mouth disease virus type O prepared from the recombinant plasmid of claim 1.

3. An antigen of a recombinant immune-boosting foot-and-mouth disease virus type O, wherein the antigen is obtained by purifying and isolating the recombinant immune-boosting foot-and-mouth disease virus of claim 2.

4. A foot-and-mouth disease vaccine composition comprising the recombinant immune-boosting foot-and-mouth disease virus of claim 2 or the antigen of the recombinant immune-boosting foot-and-mouth disease virus of claim 3.

5. A foot-and-mouth disease diagnostic kit comprising the recombinant immune-boosting foot-and-mouth disease virus of claim 2 or the antigen of the recombinant immune-boosting foot-and-mouth disease virus of claim 3.

6. A foot-and-mouth disease diagnostic method using the foot-and-mouth disease diagnostic kit of claim 5.

7. A method of preventing or treating foot-and-mouth disease using the foot-and-mouth disease vaccine composition of claim 4.
